(19) United States
(12) Patent Application Publication
(10) Pub. No.: US 2024/0395361 A1
(43) Pub. Date: Nov. 28, 2024

(54) LINKING ACTIVITY NETWORKS AS A CLINICAL COMPUTATIONAL TOOL

(71) Applicant: Regents of the University of Minnesota, Minneapolis, MN (US)

(72) Inventors: Justin Hwang, Minneapolis, MN (US); Jinhua Wang, Edina, MN (US); Hannah Elizabeth Bergom, Eagan, MN (US); Ashraf Sameh Shabaneh, Woodbury, MN (US); Abderrahman Day, Minneapolis, MN (US)

(21) Appl. No.: 18/674,630

(22) Filed: May 24, 2024

Related U.S. Application Data
(60) Provisional application No. 63/504,105, filed on May 24, 2023.

(51) Int. Cl. G16B 25/10; G06F 16/2457; G16B 30/10

(52) U.S. Cl. CPC G16B 40/00

(57) ABSTRACT

Systems, devices, and techniques are configured to determine associations between different genes, gene signatures, and gene ecosystems. In one example, a system includes processing circuitry configured to receive, from a data repository configured to store molecular abundance values for a plurality of samples, one or more matrices comprising at least one molecular abundance value for each sample of the plurality of samples, generate a first correlation matrix by performing a first statistical correlation operation between every pair of columns within the one or more matrices, generate a second correlation matrix by at least performing a second statistical correlation operation between every pair of columns within the first correlation matrix, determine, based on the second correlation matrix, a metric indicative of one or more gene ecosystems for one or more tissue types of at least one sample of the plurality of samples, and output, for display, the metric indicative of the one or more gene ecosystems.

This application claims the benefit of and priority to U.S. Provisional Patent Application No. 63/504,105, filed May 24, 2023, the entire contents of which is incorporated herein by reference.

### BACKGROUND

Knowledge of gene behavior has aided scientific discovery in deciphering various diseases including those of cancerous origin. For instance, development and progression of many cancers are associated with functional loss of tumor suppressor genes such as TP53 or functional gains of oncogenes such as MYC. Further, specific forms of gene dysregulation events are utilized as clinical predictive biomarkers or precision therapy targets in both solid and liquid tumors including genes such as ERBB2, EGFR, and BCR-ABL. High levels of Androgen Receptor Variant 7 (AR-V7) in prostate cancer, a splice variant of AR that lacks the ligand binding domain, is associated with resistance to AR-targeted therapies (ARTs). Many cancers including breast, ovarian, and prostate with mutations in BRCA1/2 are clinically actionable due to their response to PARP inhibitors.

### SUMMARY

Techniques, systems, and devices are described herein and configured for determining context specific gene behavior from sample data. For example, an Algorithm for Linking Activity Networks (ALAN) is an example computational pipeline that first constructs a gene ecosystem purely based on patient-omic data and then evaluates the interactions of all underlying gene networks. ALAN is highly compatible with current workflow managers and conventional analytical tools. More importantly, ALAN is a data-driven computational tool that compares phenotypes of genes within ecosystems. A computing system can execute this algorithm to model the gene ecosystems from one or more sample types, such as benign prostate tissue, primary prostate cancer, and metastatic castration resistant prostate cancer (mCPRC). In this manner, the system can utilize ALAN to identify expected associations of regulatory gene networks in a number of different conditions, such as prostate cancer. The system can execute ALAN to compare behavior of genes in ecosystems and thus identify novel gene signatures, gene targets, and/or mechanisms of progression or resistance seen in patients with similar conditions, such as cancer patients. ALAN can be implemented as computational tool to dissect context specific gene behavior in future informatics-based interrogations.

In one example, a system includes a data repository configured to store molecular abundance values for a plurality of samples; and processing circuitry configured to: receive, from the data repository, one or more matrices comprising at least one molecular abundance value for each sample of the plurality of samples; generate a first correlation matrix by at least performing a first statistical correlation operation between every pair of columns within the one or more matrices; generate a second correlation matrix by at least performing a second statistical correlation operation between every pair of columns within the first correlation matrix; determine, based on the second correlation matrix, a metric indicative of one or more gene ecosystems for one or more tissue types of at least one sample of the plurality of samples; and output, for display, the metric indicative of the one or more gene ecosystems.

In another examples, a method includes receiving, by processing circuitry and from a data repository configured to store molecular abundance values for a plurality of samples, one or more matrices comprising at least one molecular abundance value for each sample of the plurality of samples; generating, by the processing circuitry, a first correlation matrix by at least performing a first statistical correlation operation between every pair of columns within the one or more matrices; generating, by the processing circuitry, a second correlation matrix by at least performing a second statistical correlation operation between every pair of columns within the first correlation matrix; determining, by the processing circuitry and based on the second correlation matrix, a metric indicative of one or more gene ecosystems for one or more tissue types of at least one sample of the plurality of samples; and outputting, by the processing circuitry and for display, the metric indicative of the one or more gene ecosystems.

In another example, a non-transitory computer-readable medium comprising instructions configured to, when executed, causes processing circuitry to: receive, from a data repository configured to store molecular abundance values for a plurality of samples, one or more matrices comprising at least one molecular abundance value for each sample of the plurality of samples; generate a first correlation matrix by at least performing a first statistical correlation operation between every pair of columns within the one or more matrices; generate a second correlation matrix by at least performing a second statistical correlation operation between every pair of columns within the first correlation matrix; determine, based on the second correlation matrix, a metric indicative of one or more gene ecosystems for one or more tissue types of at least one sample of the plurality of samples; and output, for display, the metric indicative of the one or more gene ecosystems.

### BRIEF DESCRIPTION OF DRAWINGS

FIG. 1A is a conceptual diagram illustrating an example technique for analyzing individual patient samples.

FIG. 1B is a conceptual diagram illustrating theoretical connectivity, data processing, and example visualizations of an ALAN algorithm including input matrices, gene profiles, and gene ecosystems.

FIG. 2A is a graph of an example amplified genes

FIG. 2B is a graph of example gene correlation scores

FIG. 2C is a graph of example locations of genes on respective chromosomes

FIG. 2D is a visualization of overlapping genes in different gene networks.

FIG. 2E is a graph of comparisons between ALAN outputs and other gene sets.

FIG. 3A includes graphs of correlations between AR and MYC gene signatures in the ALAN network for different tissues.

FIG. 3B is a violin plot of the ALAN network signature for different genes in different tissues.

FIG. 3C is a graph of ALAN network correlation values for different cancer types.

FIG. 3D is a visualization of overlap for ALAN network signatures for different cancer types.

FIG. 4A includes violin plots of ALAN network scores for different genes in different gene ecosystems.

FIG. 4B is a conceptual diagram of gene scoring before and after treatment.

FIGS. 4C and 4D are graphs of ALAN network scores for different sets of genes pre-treatment and post-treatment.

FIG. 4E is a set of graphs for different gene signatures in distinct ALAN gene ecosystems.

FIG. 5A is a graph illustrating ALAN ecosystems that include different proteins.

FIG. 5B is a graph illustrating the distinctiveness between ERAE and ACAE in example ALAN ecosystems.

FIG. 5C is a graph illustrating correlation between different gene ALAN network scores for RSPO2 and resistant genes.

FIG. 5D is a graph illustrating correlation between different gene ALAN network scores for RSPO2 and AR.

FIG. 5E is a graph illustrating a frequency of RSPO2 alterations in prostate cancer.

FIG. 5F is a graph illustrating an amplification frequency of RSPO2 in different patients.

FIG. 6 is a block diagram illustrating an example computing system for generating ALAN gene profiles.

FIG. 7 is a block diagram illustrating various example devices that may be configured to implement one or more techniques of the present disclosure.

FIG. 8 is a flow diagram illustrating an example technique for generating an ALAN gene profile.

FIG. 9A is a string analysis of degrees of connection indicative of interation between different genes in prostate tissue.

FIG. 9B are charts of example relationships between genes exampled using ALAN outputs from different tissue types.

FIG. 10A is a graph of example ALAN profiles for example genes of interest for prostate tissue and metastatic prostate cancer tissue.

FIGS. 10B and 10C are graphs of example hierarchical clustering of different tissue samples.

FIG. 11 is an example graph of expression of example genes across a plurality of tissue sites.

FIGS. 12A, 12B, and 12C are bar graphs of different expression and scores of example genes.

FIGS. 13A and 13B are Venn diagrams of expression for different cancers at different expression percentiles.

FIG. 13C is a graph of example proliferation scores for example APUC genes compared to other genes in an overexpression screen.

FIG. 14 is a map of distances between different genes according to ALAN gene behavior.

FIG. 15 is a bar graph of relative expression of cancer related hormone receptors.

FIG. 16 includes Pearson correlations of relative expression of hormone receptors to the APUC genes.

FIGS. 17A, 17B, and 17C are correlation matrices illustrating Spearman correlations between APUC genes and hormone receptors.

FIGS. 18A and 18B are graphs of example Hallmark signatures for different status of APUC genes.

FIG. 19 illustrates plots indicating example enrichment scores and false discovery rates.

FIGS. 20A and 20B are graphs of relative rank of genes based on differential expression in different tissues.

FIGS. 21A and 21B are graphs of example hazard ratio for different biopsy sites.

FIG. 22 includes Venn diagrams of example co-expressions for different tissues at different expression percentiles.

FIGS. 23A and 23B are graphs of overall survival probabilities for prostate cancer tumors based on biopsy site.

### DETAILED DESCRIPTION

In general, the disclosure describes systems, devices, and techniques for analyzing gene activity using algorithms for living activity networks (ALAN). Aberrant gene behavior regulates cancer progression and therapeutic sensitivity. Interpretation of gene behavior improves the utility of diagnostic tools and efficacy of precision therapies. Gene behavior is governed by activity of other genes and context-specific cues including cell type, microenvironment, and prior exposure to therapy. A gene ecosystem consists of all the coalescent gene-gene interactions that occur within a context. No current informatics tools are designed to construct and then interpret gene networks in distinct ecosystems.

Knowledge of gene behavior has aided scientific discovery in deciphering various diseases including those of cancerous origin. For instance, development and progression of many cancers are associated with functional loss of tumor suppressor genes such as TP53 or functional gains of oncogenes such as MYC. Further, specific forms of gene dysregulation events are utilized as clinical predictive biomarkers or precision therapy targets in both solid and liquid tumors including genes such as ERBB2, EGFR, and BCR-ABL. High levels of Androgen Receptor Variant 7 (AR-V7) in prostate cancer, a splice variant of AR that lacks the ligand binding domain, is associated with resistance to AR-targeted therapies (ARTs). Many cancers including breast, ovarian, and prostate with mutations in BRCA1/2 are clinically actionable due to their response to PARP inhibitors. In each of these instances, however, some patients exhibit unpredictable therapeutic responses. Further interpretation of the gene networks with consideration of disease specificity or patient response may identify genomic features that could be purposed to predict response or outcomes with greater accuracy.

Many studies have accrued-omic data from tumor-derived specimens, including DNA (genomics), RNA (transcriptomics), protein/phosphoprotein (proteomics/phosphoproteomics), epigenetics (epigenomics), or even metabolites (metabolomics). These datasets can be evaluated using various informatics solutions to resolve how genes behave and are associated with clinical phenotypes. This has led to routine use of gene panels in molecular diagnostic assays to enhance patient subtyping, outcome prediction, treatment recommendations, and other diagnostic elements to understand tumor behavior. This includes the Prosigna panel PAM50 which is used for breast cancer subtyping. While classifier approaches to patient stratification are meaningful to distinguish subtypes, they are limited because DNA based panels do not encompass relationships between genes. Examination of gene networks using transcriptomic or even proteomic data will contextualize the complex features of gene behavior which can be purposed to aid in treatment decisions or for the development of novel therapies.

Artificial intelligence (AI) and machine learning (ML) techniques can be incorporated into clinical settings to examine the many modalities of -omic data for diagnostic purposes and the monitoring of treatment progression. AI and ML also have high potential to enhance information obtained from molecular diagnostic assays by deconvoluting relationships between patient outcomes, genes, and signaling pathways. Such AI/ML techniques can be used to create predictive modeling tools that naturally yield data-driven interpretations of causal factors. For example, PNET, a neural-networking algorithm, can be applied to identify somatic driver events using DNA-sequencing data from primary prostate cancer and mCRPC15. One genomic feature, MDM4 amplifications, that represented a promising precision therapy target in mCRPC can be identified in this way. The architecture of PNET was based on 3,007 pre-curated biological pathways that were not cancer-type specific. Overall, while these AI/ML approaches predict risk of occurrence from tumor samples, they include user/machine bias. This bias is because prior to analyses, users must pre-define the selectable features in order to build the model's architecture. The selectable features are often based on known gene signatures and static definitions of biological pathways. For these reasons, the interoperability and outcomes are highly contingent on pathway structures, which can be inaccurate when contexts change. Overall, an informatic driven solution that considers the context specificity could enhance the learning capacity of AI/ML tools.

While some informatics tools “classify” signaling pathways and biomarkers, understanding of gene behavior is an entirely distinct scientific objective that can benefit from a tool that evaluates biological activities of the same gene in multiple contexts. Gene networks represent a mechanism to measure how one gene behaves. Gene networks can be represented as the summation of the varying degrees of all gene-to-gene interactions using RNA-sequencing data across many patient samples. To further account for differences in gene behavior governed by all other genes, cell types, microenvironments, disease stages, and the treatment status of patients, the cross regulation of gene networks of multiple or even all genes within “gene ecosystems” can be considered. To address this limitation, an informatics tool can study gene ecosystems and all underlying gene networks within any context. This computational pipeline is referred to herein as the Algorithm for Linking Activity Networks (ALAN). Entirely dependent on the input data, ALAN acts as an informatic-based assay that measures contextual gene behavior. ALAN features can include: (1) constructs unbiased gene networks and ecosystems, 2) allows direct comparisons of informatics-based phenotypes, (3) has readouts and visualizations that are both interoperable and quantitative for user customization, and/or (4) is adaptable across distinct cancer-types, patient cohorts, and data input modalities. These utilities of ALAN overall contextualize gene behavior as a function of cancer gene ecosystems and deconvolute gene network interactions that were context and lineage specific. Using ALAN, cancer resistance mechanisms and observed changes in gene behavior in tumors that progressed towards advanced disease can be determined.

ALAN addresses three key issue in gene function discovery and clinical diagnostics in oncology. First, current diagnostic reports largely consist of single genes or panels that include oncogenes or tumor suppressors with prior knowledge. This may exclude genes with unknown or cancer tissue-specific function. ALAN can aggregate behavior of all detectable genes and yields an integrated yet simplified activity measure for individual patients and of all the relevant genes in a patient cohort. ALAN can make the multi-modal genomics integration into clinical decision making possible. ALAN can also identify custom pathways and measures activity of any gene, which many are precision targets, compared to all patients that are cataloged.

Second, in one example, ALAN has the capacity to consolidate numerous forms of large-scale data from materials collected from patients, including DNA, RNA, blood, aspirates, immune cells, etc. Third, pathway or panel construction is largely based on prior literature, laboratory experiments or even other diseases. In the context of the example of oncology, ALAN accounts for changes in behavior in tumor states including early/late stage tumorigenesis, drug response/resistance, and pre/post metastasis.

As described herein, and in one example, an Algorithm for Linking Activity Networks (ALAN) is described which is a computational pipeline that first constructs a gene ecosystem purely based on patient-omic data and then evaluates the interactions of all underlying gene networks. For example, the ALAN system can generate the gene ecosystem based on different statistical correlations, one performed on a matrix of raw molecular abundance values to generate a first matrix, and another performed on the first matrix to generate a second matrix. The first and second matrices can provide different gene relationships and gene ecosystems for different types of tissues. ALAN can be highly compatible with current workflow managers and conventional analytical tools. More importantly, ALAN can be used as a data-driven informatics assay that compares phenotypes of genes within ecosystems.

In some examples, ALAN can model the gene ecosystems of benign prostate tissue, primary prostate cancer, and/or metastatic castration resistant prostate cancer (CPRC). ALAN results can provide expected associations of regulatory gene networks in prostate cancer. For example, ALAN can identify differential and complex networks associated with the proto-oncogene MYC as prostate tumors develop, become metastatic, or in subtypes of hormone regulated cancers including breast. As another example, ALAN can probe for mechanisms that drive therapy resistance in mCRPC. This identified a novel gene ecosystem comprised of the networks of FGFRs and RSPO2 that regulated oncogenic signaling pathways including LEF1 and epithelial-to-mesenchymal transition (EMT). Altogether, ALAN can compare behavior of genes in ecosystems and thus identify novel gene signatures, gene targets, and mechanisms of progression or resistance seen in cancer patients. ALAN represents a novel tool to dissect context specific gene behavior in future informatics-based interrogations.

ALAN may include a set of algorithms and an integrated informatics tool that in parallel processes multi-modality genome-scale data from various sources including one or more of DNA, RNA, protein, methylation status, hormone/metabolic profiles, etc. The data can be collected from patients through multiple biopsy sources, such as blood or tissue. ALAN has numerous outputs that can annotate gene activity through an integration manner and in comparison with any gene in the genome. In some examples, the computational process yields results that are entirely tissue or disease specific. In some examples, the outputs can be interpretable and useful for academic scientists that are interested in discovery of gene or signaling pathways behavior in the context of basic biology, disease progression and evolution. In the clinical setting ALAN has practical uses as it allows rapid comparison of gene activities active in cancer patients through any diagnostic resource from origins such as tissue, blood, etc. In some examples, ALAN also compares individual profiles to entire patient cohorts with the same disease to rank relative activity of any gene through rank order statistics. Another clinical objective may be to include ALAN analyses in clinical reports. A workflow summarizes the data processing process. To develop the initial pipeline the system can utilize bulk or single cell RNA-sequencing data from patients.

FDA-approved or trial-based clinical parameters in precision cancer therapeutics may be currently decided based on gene mutations or dysregulation of individual genes in a panel. In contrast, ALAN can identify the gene activity as compared to every other gene detected in a patient's samples in a cohort. As described here, ALAN can be implemented as one or more of the following: a multi-modal clinical genomics data integration technique, an integrative genomics clinical report/application, a research tool, authorship, or software code that includes output the presentation in the clinical oncology and academic setting. The use of ALAN may be also applied to additional diseases in which genomic data is collected.

In academic studies, outputs from ALAN can be implemented to study gene dysregulation of signaling pathways or transcription behavior in specific tumor subtypes. Entirely custom gene signatures and profiles are generated and can be used to interpret gene function and specific gene-gene interactions at genome-scale.

In the clinical diagnostic setting, ALAN modules can be leveraged to identify hyper-activation of specific genes and pathways in tissue or blood-based biopsies from individual patients and then compare this to the result from entire cohorts. This refines patient diagnostic reports which may otherwise be based on collected big-data including WES, WTS, etc. In some examples, ALAN computes the percentile of activity compared to patients diagnosed with the same disease. ALAN can been used to identify individual resistant genes, such as RSPO2 in metastatic prostate cancer patients. ALAN can also be used to develop a new therapy resistant signature, such as 15 genes in prostate cancer patients that is observed even in single cell RNA-sequencing data.

In some examples, ALAN can be used as computational tool or as a service for consultation in areas including: novel biological and clinical discovery based on genomics, clinical decision tool for single patient diagnostics, or analytics of new patient samples in which genome-scale data is obtained.

The ALAN algorithm, which can include scripts data processing, data organization and outputs, analytics, scoring, and visualization can be presented as a custom package for informatics analysis for academic research in any disease settings in which genomic data is collected. In this manner, a computing system may implement scripts that enable the ALAN algorithm for academic research on genomic data. In some examples, the system may implement ALAN to generate panels of genes derived as resistant biomarkers that can be used as a diagnostic test for patient. In this manner, ALAN can serve as an upstream tool to nominate focused gene diagnostic panels with high-resolution protein based technology, such as O-link or Quanterix.

As described herein, the ALAN algorithm was subject to initial testing on over 1000 whole transcriptome sequencing (WTS) data from breast, ovarian, and prostate cancer patients as well as non-cancerous tissue. ALAN results confirmed prior data. For example, using WTS data alone from 200 patients, ALAN computationally identified genes that behave analogous to the androgen receptor in prostate cancer. These protein products of these genes (HOXB13, FOXA1, GRHL2, EP300, etc.) have been separately shown to bind to transcription regulatory sites using Chromatin Immuno-precipitation, crystallography approaches in prostate cancer cell lines and patient tissue. ALAN thus confirmed results that have previously taken decades using more manual investigation. ALAN also in silico predicted a recent discovery in an AR and MYC transcriptional co-regulatory interaction. As another example, ALAN identified a novel resistant gene, RSPO2, in prostate cancers, due to its similar behavior to other resistance genes from prior studies. ALAN then confirmed that RSPO2 is a resistant gene in prostate cancer models. As another example, ALAN was able to map known (cell cycle, DNA-damage, hormone transcription, etc.) or novel pathways. ALAN was implemented to identify a new pathway in metastatic prostate cancer pathways, e.g., androgen production, conversion, and uptake).

Regarding clinical utilities, ALAN can derive therapy resistant signatures based on retrospective examination of clinical samples. In one example, specifically in single cell RNA-sequenced tumors, ALAN determined that a patient that progressed on the standard of care, enzalutamide, had increased score of this signature in many of the cells post treatment.

The input and outputs of ALAN are numerous and include novel pathway discovery, gene-gene, gene-profile comparisons, signature development, and patient to cohort comparisons. ALAN can analyze steroid hormone driven cancers including breast, prostate and ovarian, but ALAN can be extended to all cancer types with WES and WTS data. In some examples, ALAN can examine profiles of risk factors in additional disease contexts (e.g., diabetes, neuro-degenerative, etc.). With other types of big data available from patients (e.g., protein, phospho-proteomics, immune cells, or steroid profiles), ALAN modules can provide outputs indicative of these interactions. In one specific example, ALAN can be used to analyze single cell RNA-seq data in a prospective clinical trials in which circulating tumor cells can be obtained from prostate cancer patients with advanced prostate cancer. ALAN can also be leveraged to investigate more types of cancer including liver and pancreatic cancers. In some examples, ALAN can be leveraged to determine therapies for patients based on genomic profiles obtained from tissue or blood-based biopsies that will include materials such as circulating tumor DNA, circulating tumor cells, hormones, immune cells.

In some examples, ALAN computes a custom signature that is entirely based on patient data in which the same gene, such as Androgen Receptor, is characterized by distinct signatures in cancers including breast subtypes, prostate and ovarian. In some examples, ALAN can produce custom gene signatures and profiles in simple matrix or visualization formats. ALAN can be configured to present findings from individual patients with cohorts of patients and presents a suggested course of clinical decision.

In some examples, the Algorithm for Linking Activity Networks (ALAN) computational pipeline can accept as input m×n matrices with m sample IDs (rows) and n molecular IDs (columns), where each cell contains a measurement of any molecular abundance such as gene expression, counts, or protein abundance. All measurements can come from distinct samples. ALAN next can serially conduct two statistical correlation operations in which all intermediate matrices are exported for quantitative comparisons and visual assessment. A rank-based Spearman's correlation can then performed between every pair of columns (molecular IDs) which generates the first correlation matrix, Matrix 1. These relational profiles can be further compared while building the second correlation matrix by performing a Pearson's correlation between every pair of columns (relational profiles) of Matrix 1 to generate Matrix 2. This subsequent correlation matrix provides a comparative functional network profile between molecular variables defined as the gene ecosystem of the input data. The system that employs the ALAN process enables data processing that is not practically performed manually or in the mind of any person. As described herein, ALAN includes the analysis of different matrices of measurements of molecular abundance (e.g., gene expression, counts, or protein abundance) and then conducts multiple statistical correlation operations on these initial and intermediate matrices. This process of correlations enables complicated relationships to be determined as described such gene networks and gene ecosystems. For at least this and other reasons described herein, a system that employs ALAN provides advantages over other techniques and provides significant improvement in the field of diagnostics and gene analysis. For example, the system may employ ALAN to identify genes, conditions, and eventually therapies for various conditions associated with one or more genes.

ALAN inputs from cancer whole transcriptome sequencing (WTS) data in some examples. Mapped expression profiles of the following studies can be included as an example: SU2C/PCF 2019 Metastatic Prostate Adenocarcinoma (n=208), TCGA Prostate Adenocarcinoma (n=493), TCGA Breast Invasive Carcinoma (Basal n=171, Luminal A (LumA) n=499, Luminal B (LumB) n=197, Her2 n=78, and Normal n=36), TCGA Ovarian Serous Cystadenocarcinoma (n=300). Transcription data from normal prostate tissue samples (n=245) can be obtained from the GTEXportal. Each cohort can be independently analyzed through the ALAN pipeline without any pre-processing or normalization. This input feature of ALAN can be valuable to both limit computational requirements to run the pipeline and to ensure that the data is not over normalized which can result in erasing true biological events.

ALAN Matrix classifications can include gene correlations between any two genes, gene profile correlations, gene network correlations, gene network signatures, gene ecosystem, or gene ecosystem signatures. Each of these matrix classifications may be a metric indicative of the gene expression for a tissue type of one or more samples. From Matrix 1, the value in each individual cell based on the row and column ID represents a gene correlation between any two genes. Entire columns in Matrix 1 of a single gene-of-interest (GOI) were defined as an “ALAN gene profile.” Entire columns in Matrix 2 of a single gene-of-interest (GOI) were defined as an “ALAN gene network” where each value represents the similarities of a gene profile as compared to all other gene profiles. Two or more ALAN gene networks, when deemed statistically similar, were defined as an “ALAN gene ecosystem.”

ALAN gene profile correlations. The degree of correlation between two ALAN gene profiles are directly obtained from in individual cells within Matrix 2 based on row and column ID. These individual values were defined as an “ALAN gene profile correlation.”

ALAN gene network correlations. Pearson correlations can be utilized to further associate ALAN gene networks of two or more GOIs based on all row IDs and values from two or more columns from Matrix 2.

ALAN gene network signatures. The subset of a gene network for any GOI exhibiting correlations above 0.7 unless otherwise noted can be defined as an “ALAN gene network signature”. This list of genes was used to determine an ALAN network score for any other ALAN network or using values extracted from Matrix 2.

ALAN gene ecosystem and ALAN gene ecosystem signatures. Based on the previous definition of ALAN gene networks or network signatures from columns in Matrix 2, “ALAN gene ecosystems” are defined as any group of genes with highly similar ALAN gene networks. The average of all values from their ALAN gene networks can be considered as the ALAN gene ecosystem. To define the “ALAN gene ecosystem signature”, the averaged gene networks from a gene ecosystem with values above 0.7 were included as the subset of row IDs. This list of genes was used to determine an ALAN network score for any other ALAN network or ALAN ecosystem using values extracted from Matrix 2.

Individual Patient Analysis and Scoring. One mCRPC patient from a prior study in which single cell RNA-sequencing was performed on paired biopsies from both before and after enzalutamide treatment was identified. Of the data, transcripts per million (TPM) were determined for genes in ALAN ecosystems and scored them in all tumor cells by summing the z-scores of the TPM values and scaling the aggregate sums from 0-100.

Gene Set Enrichment Analysis (GSEA). Pre-ranked GSEA were conducted to depict enrichment of ALAN outputs using Hallmark gene sets and C6 oncogenic signatures to obtain net enrichment score (NES) based on FDR of 0.019. The ranked list of genes with ALAN scores were obtained from either Matrix 1 or 2 as one example.

GISTIC 2.0 calling of the AR focal amplicon. Focal amplifications can be identified in 493 primary prostate adenocarcinomas from the TCGA Pan Cancer (http://firebrowse.org/) study to identify the genomic regions that harbor frequent amplifications in prostate cancer (PCA).

HUGO (HGNC) mapping of chromosomal locations-Waterfall Plot. The HGNC Multi-Symbol Checker can be utilized to identify the chromosomal locations of 19,000 genes sequenced in the SU2C 2019 mCRPC study.

Consolidation of Surfaceome and Secretome. To create the list of surface proteins consolidated into the “Surfaceome,” datasets were integrated from the Cell Surface Protein Atlas, which includes 1492 protein IDs and the Human Protein Atlas, which includes 5318 protein IDs, for a total of 5890 cell surface proteins. The list of 1678 secreted proteins used to define the “Secretome” can be obtained from the Human Protein Atlas.

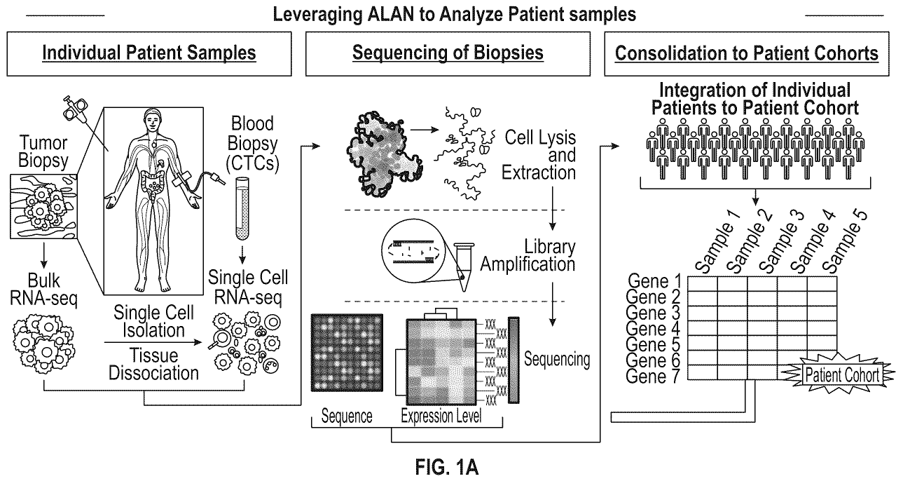

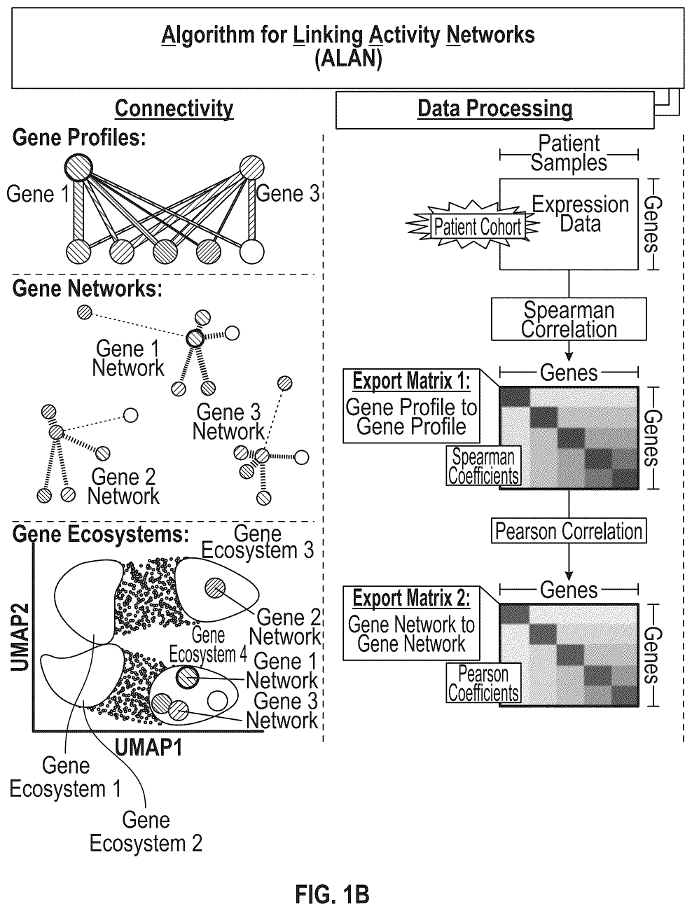

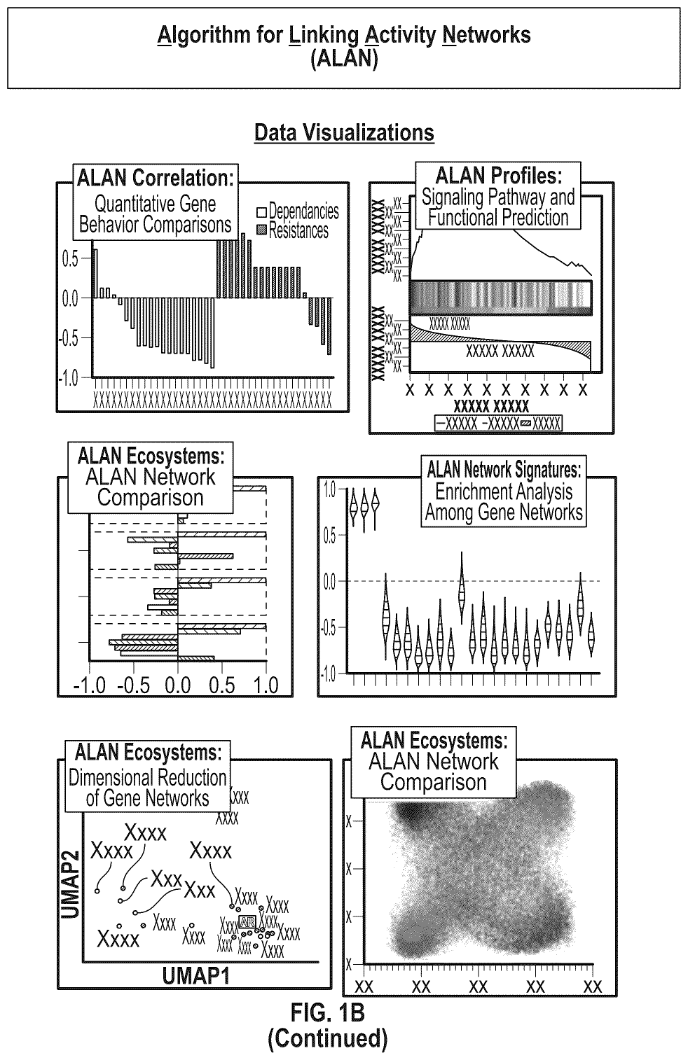

FIG. 1A is a conceptual diagram illustrating an example technique for analyzing individual patient samples, and FIG. 1B is a conceptual diagram illustrating example visualizations of gene network. FIGS. 1A and 1B describe leveraging ALAN to analyze patient samples, such as individual patient samples. Some steps of the process can include visualization of the collection process of individual patient samples from either tissue or blood-based biopsies, sequencing of biopsies, and the depiction of the preparation process for bulk or single cell sequencing. The process can also include consolidation to patient Cohorts, as shown by visualization of the integration process of individual patient samples into patient cohorts from public datasets where cohort expression profiles are generated from individual patient biopsies. The Algorithm for Linking Activity Networks (ALAN), can include a workflow shown which depicts the ALAN pipeline which can include data processing techniques such as generating different matrices from source gene expression data for different patient samples, connectivity between genes, gene networks, and gene ecosystems, and data visualizations of any types of metrics of the gene connectivity results, as shown in FIG. 1B.

ALAN can be leveraged to conduct deep interrogations downstream of conventional diagnostic tools including both bulk and single cell RNA-sequencing technologies, as shown in FIGS. 1A and 1B. These outputs from ALAN generally yield RNA expression profiles in distinct units (TPM, FPKM, RPKM, etc.). ALAN can be adapted to receive input of any form of data in a tabular matrix format, as one example. At this stage, the ALAN computational pipeline fully establishes multiple layers of gene connectivity. In some examples, ALAN constructs gene profiles by evaluating every gene-to-gene expression association among all detectable genes, in all patient samples, using a rank-based association method. The gene networks can then be established from all existing gene profiles using a second association based on the continuous values. As part of the pipeline, ALAN generates data matrices in which the values inform similarity measurements of gene profiles and networks represented as informatic based gene phenotypes, as well as accompanying data visualization tools to compare gene profiles and networks, such as the visualizations in FIG. 1B. The aggregate of multiple gene networks with statistical significance reflects an ALAN gene ecosystem (FIG. 1B). These gene ecosystems can be utilized to analyze patient data from the perspective of cancer progression and therapy resistance. In other words, one or more metrics indicative of gene expression as obtained through the ALAN system can be used to diagnose disease or conditions and/or generate or modify a therapy plan for a patient or for a type of tissue.

ALAN outputs can predict critical AR activity in mCRPC. For example, the Androgen Receptor (AR) is critical for the development of prostate cancer (PC) and remains a critical target in both metastatic prostate cancer (mPC) and metastatic castration resistant prostate cancer (mCRPC). Current standard-of-care agents for mCRPC inhibit synthesis of AR ligands or signaling of AR. While these AR-inhibiting therapies (ART) are initially effective, a subset of patients inevitably develop resistance to these therapies. AR mutations, amplification, or overexpression remain prevalent in patients that develop resistance to multiple therapies which reinforce that AR functions remain critical in advanced cancers.

To examine the gene networks in prostate cancer, in one example, data was obtained from 946 individuals in which whole transcriptome sequencing (WTS) was conducted on normal tissue, primary cancer, and metastatic tissue. The data were obtained through the Genotype Tissue Expression (GTEX) project, The Cancer Genome Atlas (TCGA), and Stand Up to Cancer 2019 (SU2C 2019).

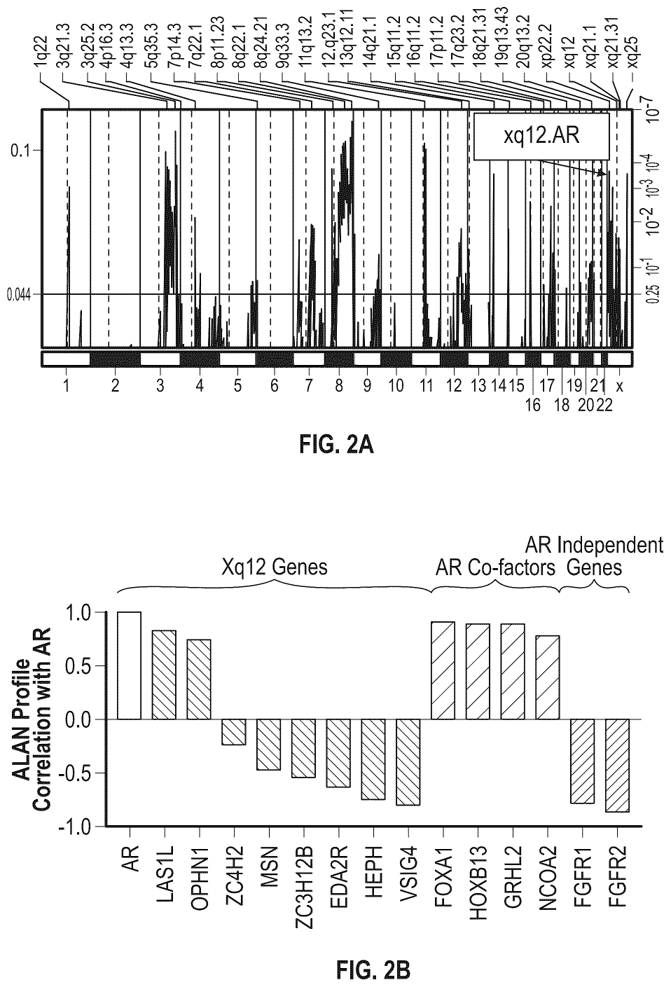

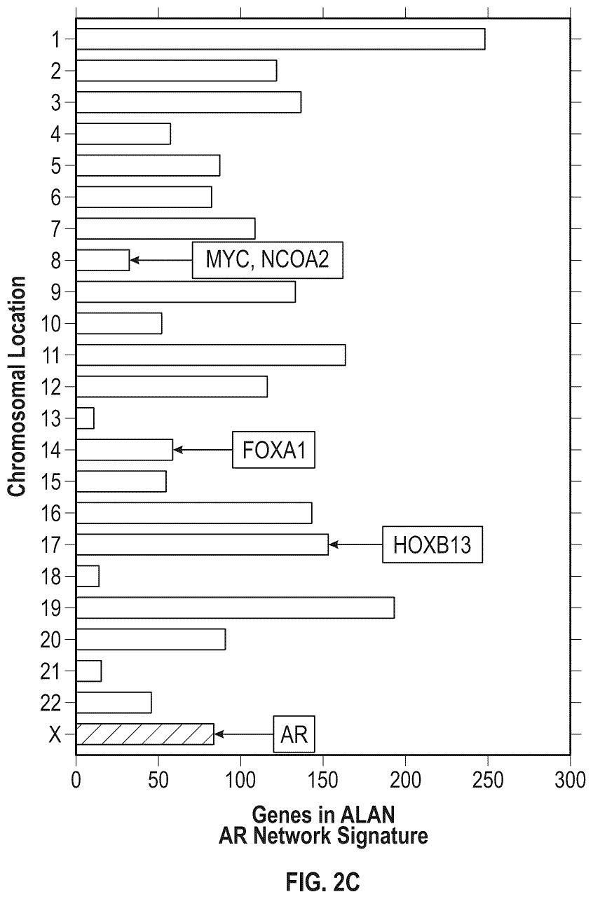

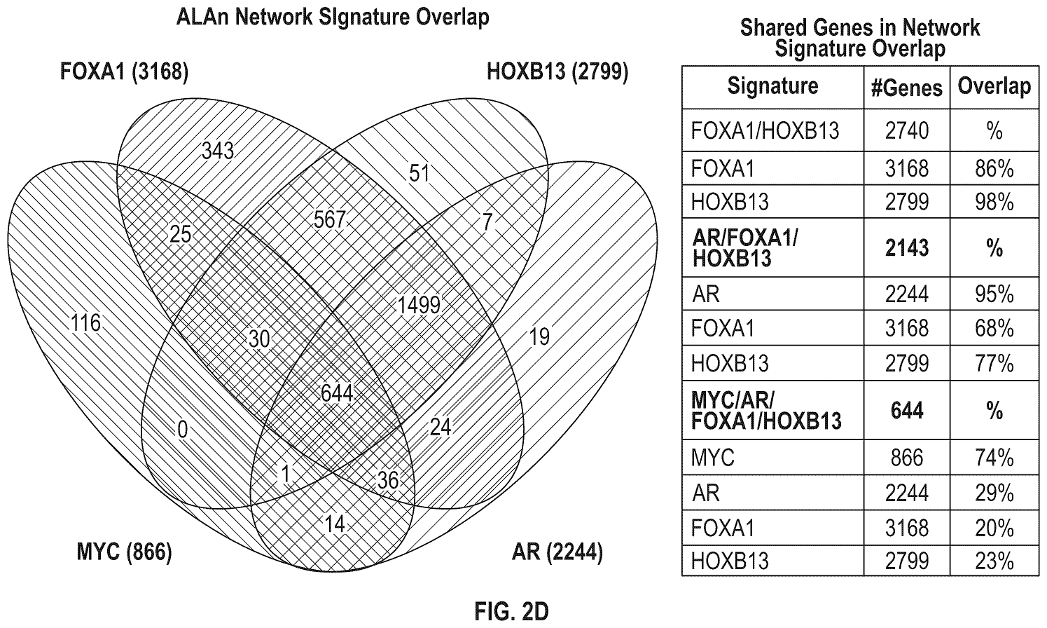

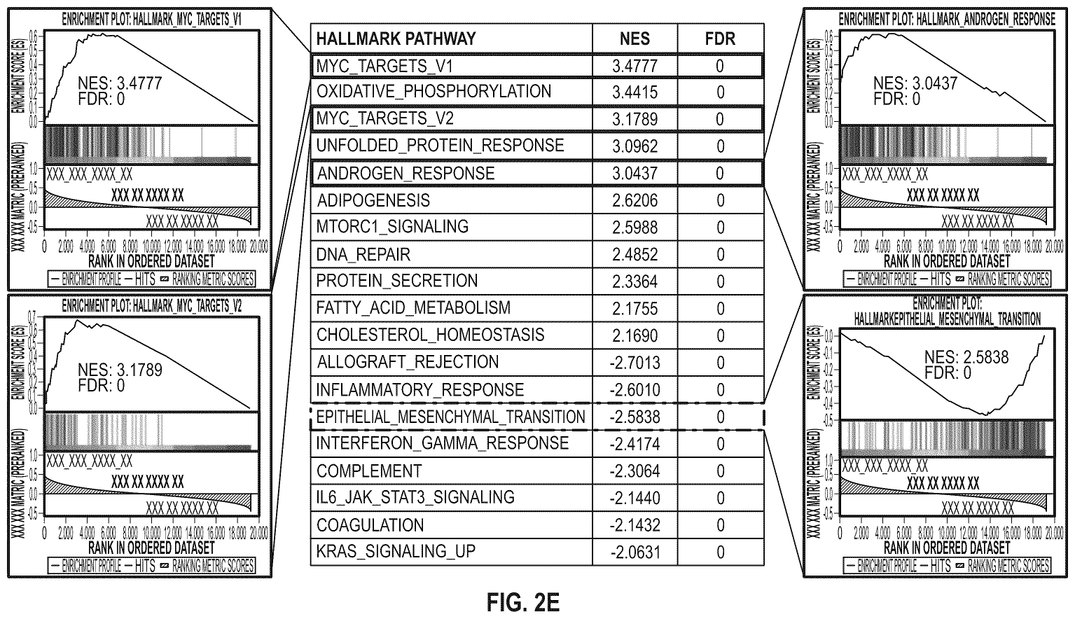

FIG. 2A is a graph of an example amplified genes, FIG. 2B is a graph of example gene correlation scores, FIG. 2C is a graph of example locations of genes on respective chromosomes, FIG. 2D is a visualization of overlapping genes in different gene networks, and FIG. 2E is a graph of comparisons between ALAN outputs and other gene sets.

In the examples of FIGS. 2A-2E, the functionality of ALAN was tested to interpret projected interactions between AR and known co-factors in mCRPC patients, of which many are intrachromosomal. To map co-amplified genes of AR, outputs from GISTIC 2.0 were utilized to identify the recurrent focally amplified region of AR in 492 primary prostate cancer tumors as shown in FIG. 2A, which shows GISTIC2 analysis of primary prostate adenocarcinomas highlighting the focal amplification of AR (Xq12, n=9) as a recurrent copy number gain (TCGA PRAD, n=492). AR resides in a focal amplicon on Xq12 with 9 additional genes where only 8 of the 9 genes were detected in this cohort. ALAN was first examined to determine if it could predict known AR interactions and if genomic interactions, such as focal amplifications, were indicative of similar gene profiles and gene networks in 208 mCRPC patients. As indicated by their ALAN profile correlation score of close to 1.0, the transcription factors HOXB13 and FOXA1, both known co-factors of activated AR, had similar ALAN profiles. However, of the genes on Xq12 within the same focal amplicon as AR, the majority had negative correlation scores (FIG. 2B, which shows ALAN profile correlations with AR are shown for other Xq12 genes (gray), AR co-factors (orange), and AR independent genes (blue)). This reveals that while AR, HOXB13, and FOXA1 had similar gene behavior as defined by aggregate expression patterns across the transcriptome, this behavior was dissimilar to other genes located within the AR focal amplicon and suggests that they are functionally dissimilar to AR in mCRPC. To explore additional intrachromosomal interactions with AR, the chromosomal location of all 2,244 genes within the ALAN AR network signature were categorized. Of all the genes in the ALAN AR network signature, only 3.8% were located on the X chromosome. However, the ALAN AR network signature contained the co-factors FOX1 (14q21) and HOXB13 (17q21), the proto-oncogene MYC (8q24), and coactivator NCOA2 (8q13), as shown in FIG. 2C, which illustrates the chromosomal location of genes in the ALAN AR network signature. Highlighted genes have implications with AR in prostate cancer such as transcription factors MYC (8q24), FOX1 (14q21), HOXB13 (17q21), and transcriptional co-activator NCOA2 (8q13). In this regard, ALAN predicted known interactions with AR in mCRPC including association with transcription factors HOXB13, FOX1, and the proto-oncogene MYC. These results indicate that these informatic based phenotypes generated by ALAN are powered to identify intrachromosomal relationships between genes as a factor of gene behavior in mCRPC patients.

The networks of these genes in mCRPC were further explored by examining the similarities of their ALAN network signatures, as shown in FIG. 2D which includes a visual depiction of ALAN network signature overlap between AR (red), MYC (yellow), FOX1 (orange), and HOXB13 (orange) and their percentage of shared genes. As depicted in the Venn diagram of FIG. 2D, AR shared 95% of the genes in its 2,244 ALAN network signature with FOX1 and HOXB13, whereas 74% of the 866 genes in the MYC ALAN network signature were shared with AR, FOXA1, and HOXB13. This indicates that these genes not only have similar gene profiles (FIG. 2B), but also have similar gene networks (FIG. 2D) indicating that they reside in the same gene ecosystem in mCRPC. These results support the assertion that ALAN can identify these previously known and essential critical gene networks in mCRPC through transcriptomic data.

The ALAN outputs can be integrated directly into current genomic analytical tools, such as Gene Set Enrichment Analysis (GSEA). To evaluate the AR ALAN profile through conventional means, GSEA analyses were conducted on ALAN outputs from the mCRPC samples (FIG. 2E, which illustrates GSEA Enrichment Plots with NES and FDR statistics of the AR ALAN profile and various Hallmark Gene Signatures derived from ALAN Matrix 1). Of the fifty hallmark gene sets from MSigDB, the ALAN AR profile was associated with the Androgen response signature (NES, =3.04, FDR, =0) and two MYC Hallmark gene sets (MYC Targets V1 and V2, NES=3.48, 3.18, FDR=0, 0). Additionally, the AR ALAN network was de-enriched of signatures, such as epithelial-to-mesenchymal transition (EMT, NES=−2.58, FDR=0), which has been recently identified as an upregulated signaling pathway in mCRPC patients that have developed resistance to ART. While the AR ALAN profile is enriched of pathways involving MYC and androgen signaling, it is de-enriched of pathways associated with metastasis and therapeutic resistance such as EMT. This suggests that ALAN is identifying AR signaling and therapeutic resistance as potentially separate mechanisms. These observations bolster the biological findings and display both the value and ease of integrating ALAN outputs with current informatics analyses (FIG. 2E).

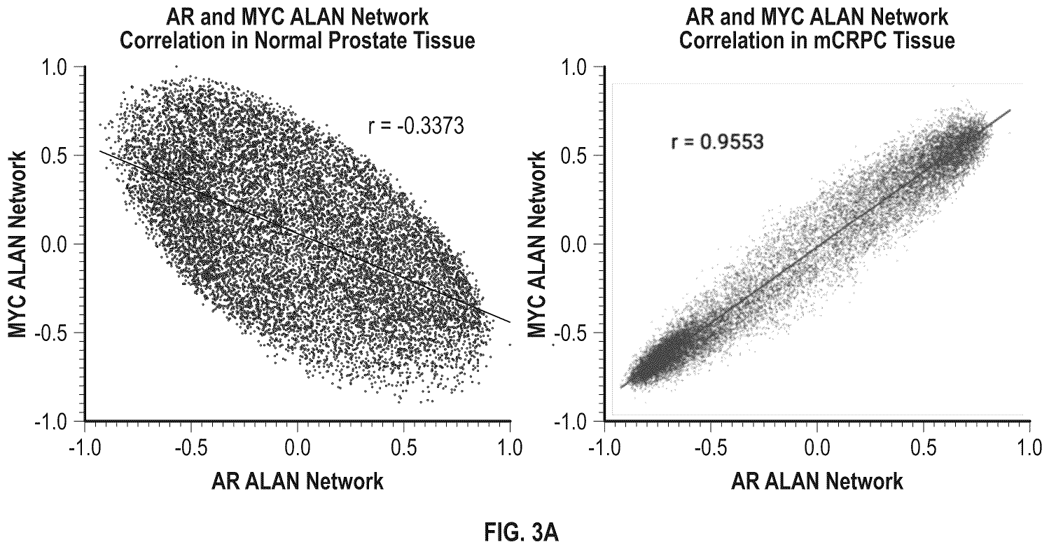

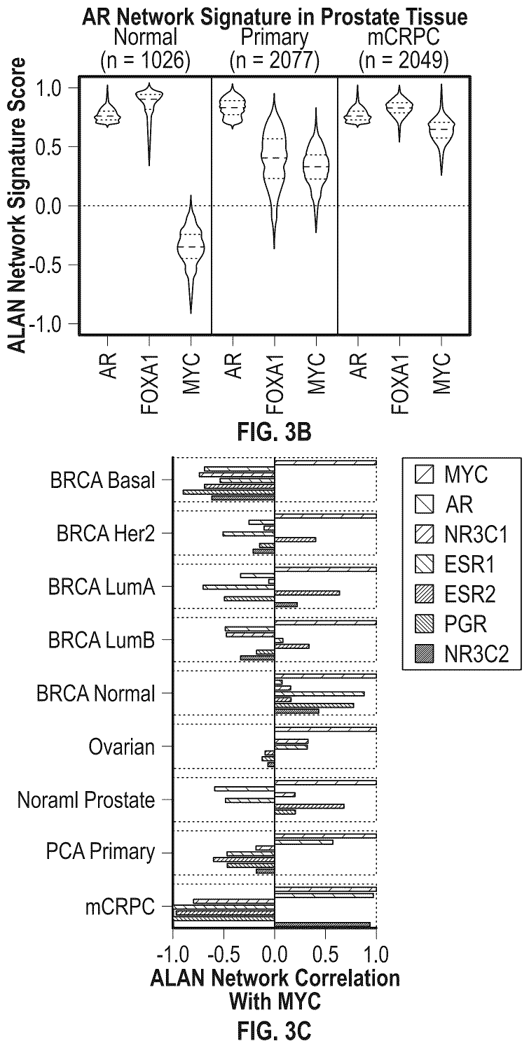

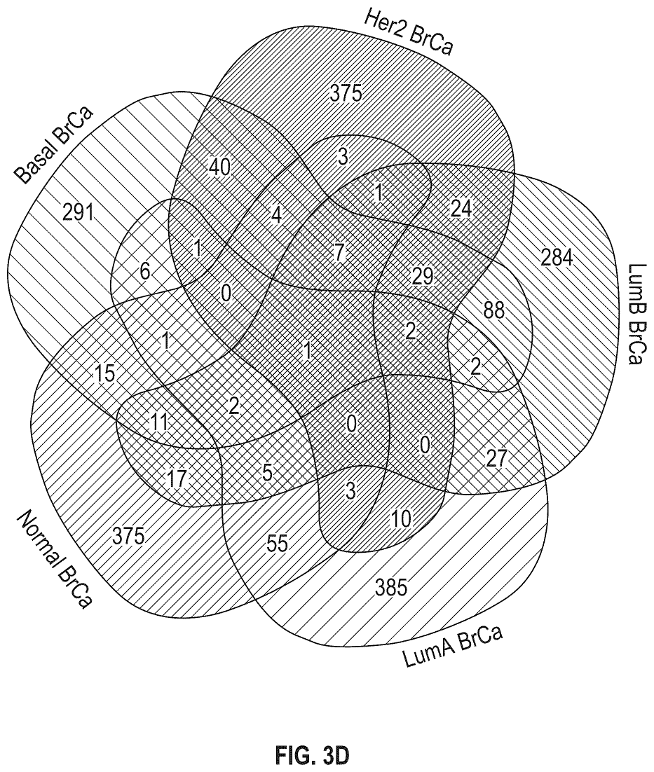

As shown in FIGS. 3A-3D, ALAN can also be used for mapping of MYC Activity in Prostate Tumor Progression and Subtypes of Breast Cancer. FIG. 3A includes graphs of correlations between AR and MYC gene signatures in the ALAN network for different tissues. FIG. 3B is a violin plot of the ALAN network signature for different genes in different tissues. FIG. 3C is a graph of ALAN network correlation values for different cancer types. FIG. 3D is a visualization of overlap for ALAN network signatures for different cancer types.

Functional gains and overexpression of the oncogene MYC have been implicated in the development and progression of many cancers. Previous literature has indicated that MYC begins to associate with AR exclusively in mCRPC. Understanding the context specific nature to gene behavior, ALAN was utilized to examine gene behavior evolution of MYC and AR across tumor stages by examining the changes in their ALAN networks as a function of disease progression. The similarity of the AR and MYC ALAN networks was analyzed by performing an ALAN gene network correlation in normal prostate tissue compared to mCRPC (FIG. 3A). In FIG. 3, an ALAN network correlation is shown for AR and MYC in both normal prostate tissue and mCRPC as visualized with linear regression statistics. Each dot represents an individual gene where its x- and y-coordinates represent the ALAN profile correlations with either AR or MYC respectfully. The global associations of the MYC network were negatively correlated with the AR network in normal prostate tissue (FIG. 3A, Left; r=−0.3373 p-value <0.0001) but robustly aligned to the AR network in mCRPC (FIG. 3A, Right; r=+0.9553 p-value <0.0001). This indicates that the informatic based phenotype of AR and MYC in mCRPC is highly similar and in normal prostate tissue is dissimilar. To further this analysis, we compared the ALAN gene networks of AR, FOX1, and MYC using the AR ALAN network signature. As prostate tissue progressed from normal, to primary, to mCRPC, a gradient of increasing signature scores was observed between MYC and the AR ALAN network signature with the greatest association in mCRPC. This data indicates that while these genes share an ecosystem in mCRPC, ALAN directly visualizes these changes in networks as an evolution of gene behavior (FIG. 3B, where the ALAN AR network signature is depicted using a violin plot for the ALAN networks of AR (red), FOX1 (orange), and MYC (yellow). Individual genes in the indicated signature are compared in normal, primary, and mCRPC where the number of genes in each signature is indicated (n)). Altogether, these ALAN analyses allowed us to observe global changes in networks and ecosystems for the same genes across tissue with expectedly distinct histopathological behavior.

These initial observations consolidated interactions between MYC and AR in mCRPC, but other cancer types including breast and ovarian are also driven by aberrant signaling of steroid hormone receptors (SHR) and the proto-oncogene, MYC. To investigate the association of MYC and SHRs in other endocrine driven cancers, ALAN was utilized to examine their gene networks. Transcription data was obtained from The Cancer Genome Atlas (TCGA) on samples of ovarian cancer and the five PAM50 molecular subtypes of breast cancer (Basal, HER2, Luminal A, Luminal B, normal-like). Of the outputs, the ALAN network correlation with MYC and each SHR was analyzed, as shown in FIG. 3C. In FIG. 3C, the ALAN network correlation with MYC is visualized using a waterfall plot for the networks of MYC (yellow), AR (red), NR3Cl-Glucocorticoid Receptor (light blue), ESR1—Estrogen Receptor (light pink), ESR2-Estrogen Receptor (dark pink), PGR—Progesterone Receptor (blue), and NR3C2—Mineralocorticoid Receptor (gray). Quantitative ALAN network correlation values are determined within various cancer types including five subtypes of breast cancer (BrCa; Basal, Her2, Luminal A (LumA), Luminal B (LumB), and Normal), ovarian, normal prostate, primary prostate, and mCRPC

Interestingly, the network correlation with MYC and SHRs across all the tissue types were unique and exhibited distinct degrees of overlap, with ESR2 demonstrating a consistent positive association with MYC in all breast cancer subtypes except Basal. To further investigate if the MYC ALAN networks were divergent within the subtypes of breast cancer specifically, the MYC network signature of each subtype was analyzed. Each signature was built using the top 500 genes associated with MYC in that subtype. In this cross-subtype comparison, 82.6% of all genes across the five ALAN MYC network signatures were unique to one subtype and only 2.8% of genes (69 total including MYC) were shared by at least three subtypes (FIG. 3D). The overlap between MYC ALAN network signatures across five subtypes of breast cancer (Basal, Her2, LumA, LumB, and Normal) are visualized using a Venn diagram in FIG. 3D. Of those 69 genes, three of them, EIF3E, MLLT6 and MYC, are indicated as cancer causing genes by The Sanger Institute. These results indicate that while MYC is a critical driver in many cancers, its biological function may be divergent due to the unique and specific interactions within each SHR-driven cancer subtype. Therefore, the use of ALAN to identify these unique gene ecosystems in additional genes and cancers can be used to deconvoluting context specific gene behavior which be leveraged for more personalized medicine approaches.

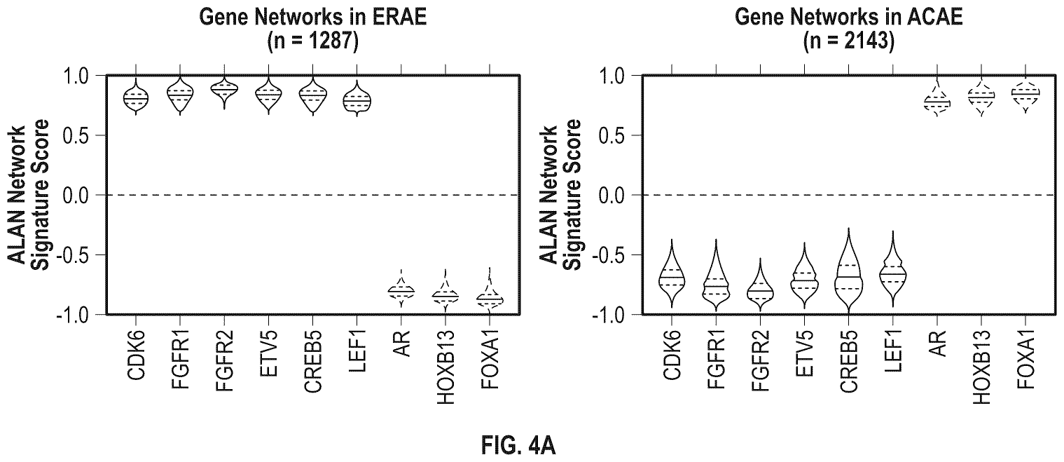

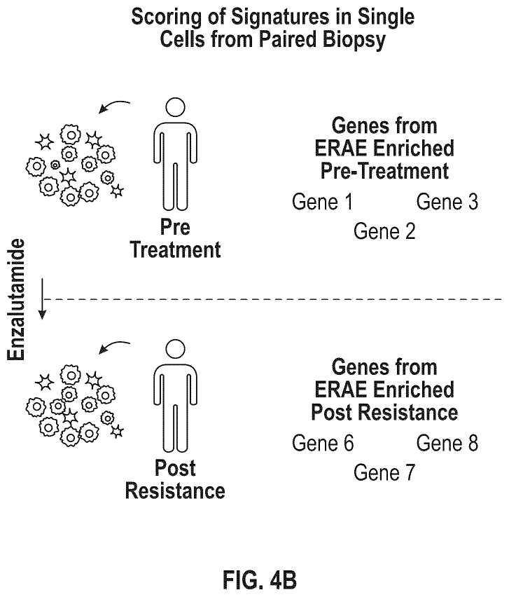

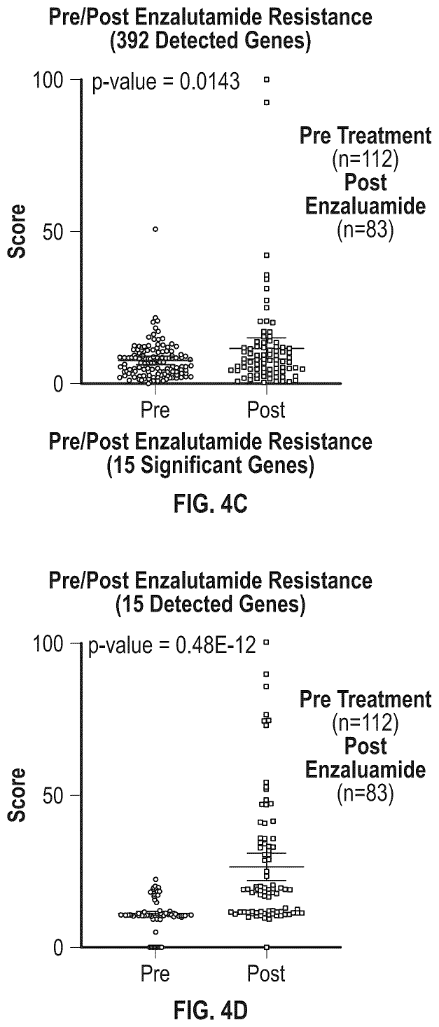

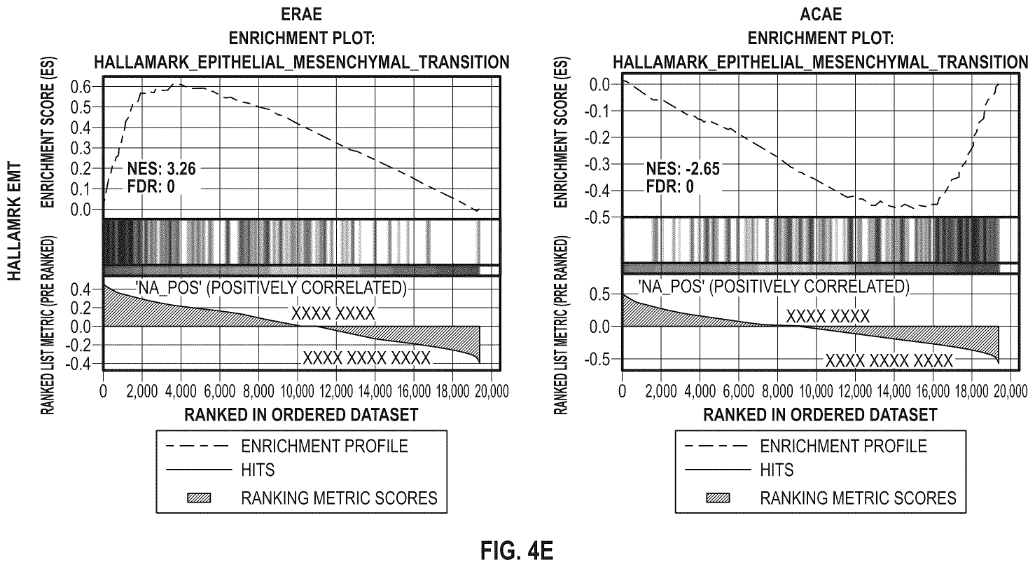

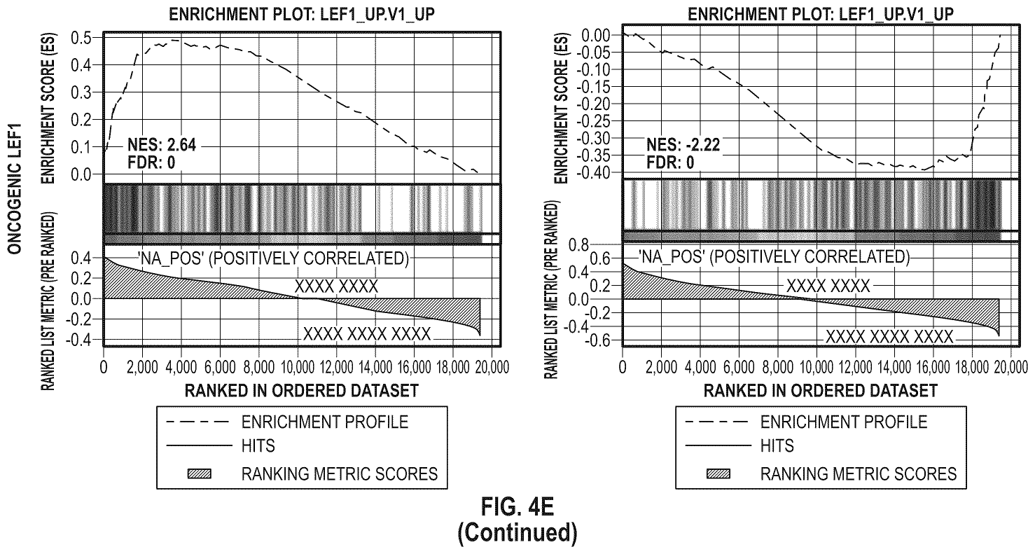

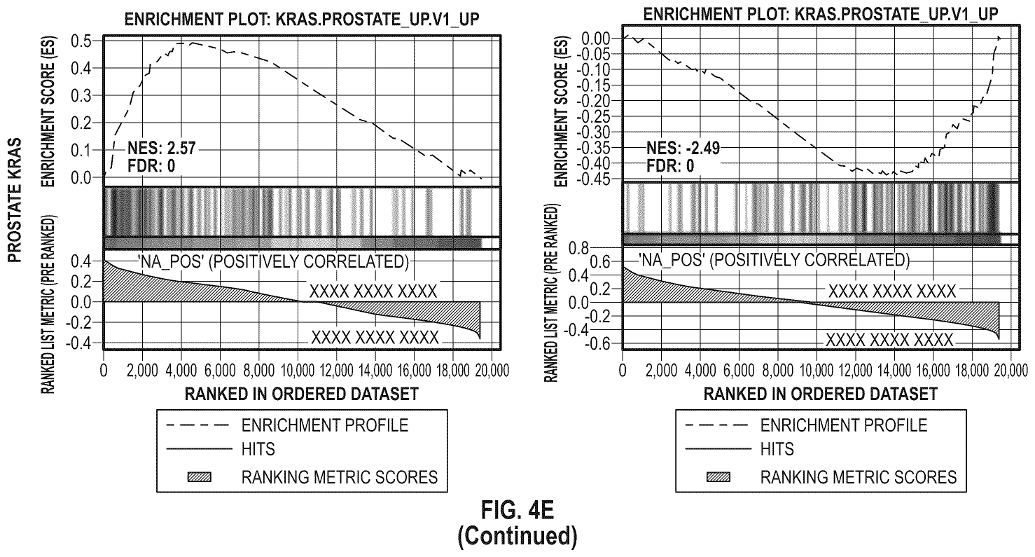

ALAN can also leveraged for the identification of genes and pathways that predict resistance. In addition to examining reported interactions, we sought to elucidate critical biology by aggregating the consistent interactive patterns of multiple ALAN gene networks, or their ALAN gene ecosystems. FIG. 4A includes violin plots of ALAN network scores for different genes in different gene ecosystems. FIG. 4B is a conceptual diagram of gene scoring before and after treatment. FIGS. 4C and 4D are graphs of ALAN network scores for different sets of genes pre-treatment and post-treatment. FIG. 4E is a set of graphs for different gene signatures under different environments.

The gene ecosystems of AR and co-factors (HOXB13, FOXA1) as well as several genes that regulated enzalutamide resistance including CDK6, FGFR1/2, ETV5, LEF1, CREB5, were examined. Upon examining the Enzalutamide Resistance ALAN Ecosystem (ERAE, genes=1287) and the AR and Co-factors ALAN Ecosystem (ACAE, genes=2143), the genes within each ecosystem were observed to be co-segregated but that the two ALAN ecosystems were divergent and had 0 overlapping genes (FIG. 4A, where the ERAE (left) and ACAE (right) are depicted using a violin plot for the ALAN networks in blue (CDK6, FGFR1, ETV5, CREB5, and LEF1) and orange (AR, HOXB13, and FOX1)). It was also examined if the ERAE genes could reflect the clinical response in an individual mCRPC patient towards treatment progression (FIG. 4B). FIG. 4B is a workflow depiction of using a unique ALAN gene ecosystem signature for scoring cells from an individual patient with paired biopsies. In this example, scRNA seq data obtained from paired biopsy samples from one patient pre-enzalutamide treatment and post-therapy resistance was examined. Due to the sparse nature of scRNAseq data, not all genes that were sequenced had detectable count values. Therefore, all genes that were detected in both paired biopsies from the ERAE (392 genes) were first scored and found a statistically significant increase across the tumor cells from the enzalutamide-resistant biopsy (FIG. 4C). In FIG. 4C, scores were computed using the ERAE in all tumor cells that were pre-treatment (n=112) or post Enzalutamide resistant (n=83) in one mCRPC patient. 392 of 1288 genes were detected in the scRNA-seq data. The statistical significance (p-value) was computed using a Student's t-test.

It was subsequently noted that 15 of the 392 genes demonstrated the most robust statistical significance (p-value=1.48E-12) in this patient post treatment (FIG. 4D). For FIG. 4D, scores are depicted for all tumor cells from FIG. 4C using the 15 genes with the greatest expression increases in the post resistant sample. This result demonstrates that ALAN is powered to generate unique gene signatures and potentially gene panels based on clinical response and resistance status. These findings may also be consistent for other similar patients. To further interrogate the biology associated with the ACAE and ERAEs in mCRPC patients, GSEA was performed and found that gene signatures including EMT, oncogenic LEF1, and several tissue-associated KRAS signatures (NES=3.26, 2.64, 2.57; FDR=0.0) were positively associated with the ERAE (FIG. 4E). As shown in FIG. 4E, aggregate ERAE and ACAE are analyzed and depicted through GSEA

Enrichment Plots. Hallmark EMT, Oncogenic LEF1 and Prostate KRAS are shown with NES and FDR statistics. Conversely, the ACAE exhibited negative enrichment of these same signatures. These oppositional ecosystems further bolster the previous result (FIG. 2E) and indicates that ALAN identifies these groups of genes, using both gene networks and now gene ecosystems, as potentially separate mechanisms in mCRPC patients. Overall, the two ALAN gene ecosystems exhibited dichotomous relationships, as characterized by their underlying gene networks and signaling pathways.

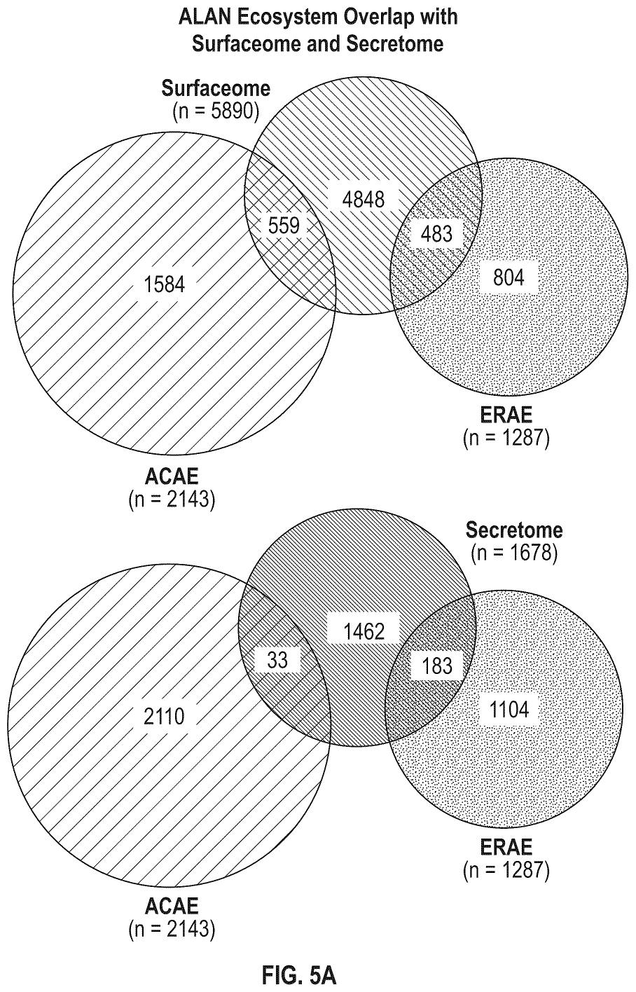

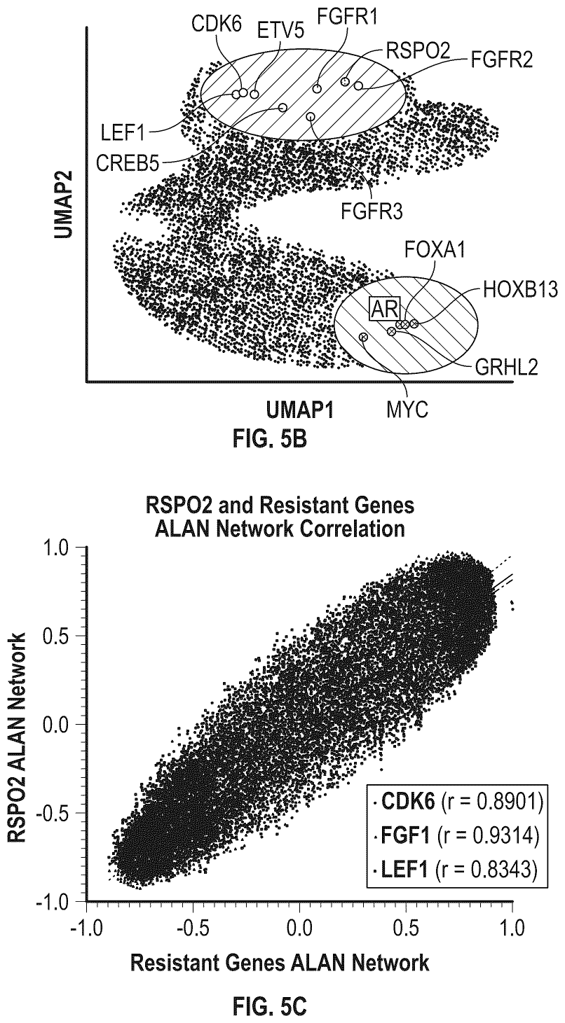

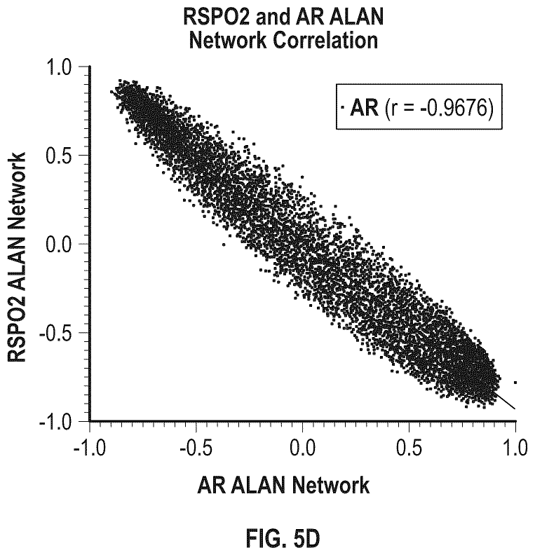

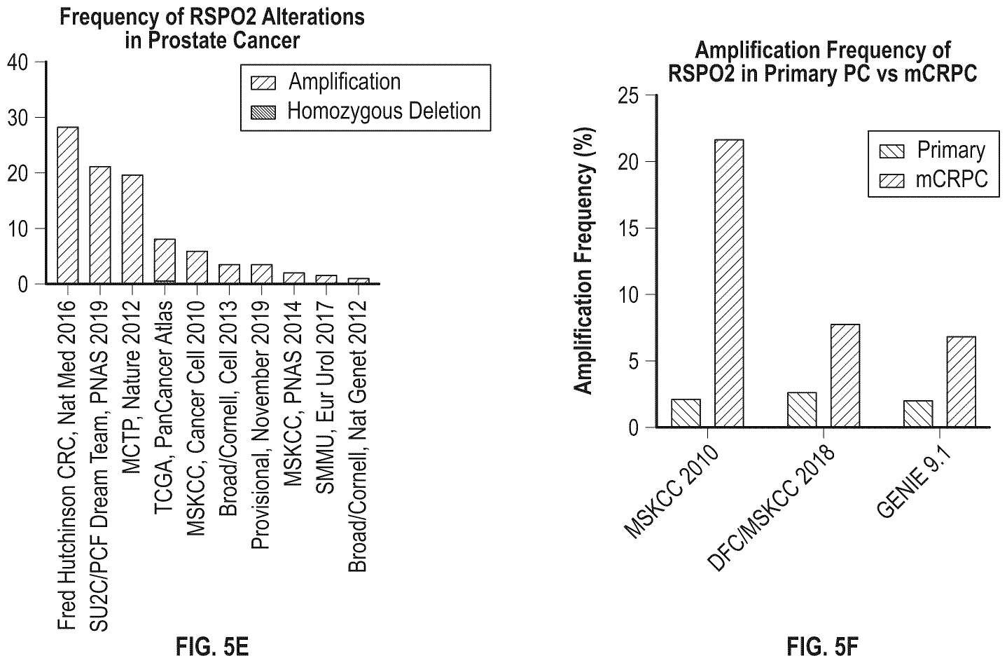

In some examples, the ALAN resistance gene ecosystem nominates potential future therapeutic targets. FIGS. 5A-5F provide illustrations of how to identify one or more genes with similar functional networks. In one example, ERAE can predict alternative targets that could be used against mCRPCs that develop resistance to ART. In this example, surfacesome and secretome proteins were examined for future antibody (neutralizing, radio-labeled, drug-conjugates) or immune-cell (Natural Killer, T-Cell engagers, CAR-T cells) therapies. Upon integrating ERAE and ACAE with proteins found in the Surfacesome (FIG. 5A, top) or Secretome (FIG. 5A, bottom), these ecosystems again included distinctly different proteins. In FIG. A, the gene overlap of ERAE (blue) and ACAE (orange) with Surfaccome (top) and the Secretome (bottom) are depicted using Venn Diagrams where the number of genes in each group (n) and the number of overlapping genes are depicted.

To further illustrate the distinctiveness of the ERAE and ACAE, their relative degree of association with each other and all other genes in mCRPC via dimensional reduction on an x-y-plane through Uniform Manifold Approximation and Projection are illustrated in FIG. 5B. In FIG. 5B, the UMAP plot depicts ALAN gene ecosystems in which specific gene networks of known biological activity are highlighted. Both the ERAE (blue) and ACAE (orange) are shown as well as nominated resistant gene RSPO2 (light blue). Notably, RSPO2 was identified within the ERAE. In some examples, mCRPCs harbor amplification or expression-driving fusion events of RSPO2, which encodes a secreted WNT signaling enhancer 26. The RSPO2 network overall had a positive ALAN network correlation with other enzalutamide resistant gene networks including CDK6, LEF1 and FGFR1, as shown in FIG. 5C (all p-value <0.0001), while we observed an opposing ALAN network correlation within the AR ALAN network, as shown in FIG. 5D (p-value <0.0001). In FIG. 5C, The ALAN network correlation of the RSPO2 network with ALAN networks for CDK6 (blue), FGFR (light blue), and LEF1 (purple) is depicted using ALAN cloud plots with linear regression statistics shown. In FIG. 5D, the ALAN network correlation of the RSPO2 network with the AR (red) ALAN network is depicted using ALAN cloud plots with linear regression statistics shown. This result indicates that RSPO2 is behaving more similar to genes within the ERAE than genes within the ACAE in mCRPC patients.

In addition, it was also confirmed the genomic observations of previous studies in which RSPO2 is a recurrently amplified gene with limited deletions in prostate cancers, as shown in FIG. 5E. FIG. 5E illustrates that alteration frequency of RSPO2 amplifications and homozygous deletions in various prostate cancer (PCA) datasets. Further, in studies that sampled both primary and mCRPC patients, RSPO2 amplifications were observed at increased rates in the mCRPC samples, as shown in FIG. 5F. FIG. 5F displays a comparison of RSPO2 amplification frequency in primary PCA vs mCRPC patients in MSK2010, DFCI/MSKCC 2018, and GENIE 9.1 datasets. Altogether, these findings indicate that ALAN ecosystems can be utilized to identify more genes with similar functional networks, and that this approach may yield targets that are on the surface or are secreted in mCRPCs.

The current scientific environment demands improved informatics-based approaches to research gene behavior. Existing tools utilize prior knowledge of signaling pathways and gene interactions to define their algorithms. In laboratory or informatics settings, gene behavior is regulated by other genes, as well as contexts including cell type, microenvironment, treatment status, etc. However, no current tools have been developed to directly address all these differential regulatory features. As described herein, ALAN is a tool to study adaptive gene ecosystems, to provide user-friendly analytical tools, and to compare gene phenotypes. ALAN can this be used to diagnose conditions or diseases, or even determine or adjust therapy plans for one or more patients. In some examples, ALAN first constructs gene ecosystems purely based on cohorts of patient data. ALAN also includes quantitative tools to measure all possible gene networks which allows for direct comparisons of gene behaviors in the same or distinct ecosystems. Since ALAN can recognize all intricate gene-to-gene interactions across multiple stages of a cancer, or even across distinct cell types, ALAN can avoid the conventional and static definition of gene pathways in cancer. ALAN can also be utilized by a system to construct gene ecosystems that promote therapy resistant prostate cancer. This identified new sets of promising gene targets and signatures. The demonstration of these utilities indicate ALAN represents a mechanism to improve current informatics modeling of cell signaling, gene systems, or machine learning.

On a technical level, ALAN can be compatible with many platforms including existing pipelines and pathway enrichment tools. Due to the rank-based conversion in its initial analyses, any form of mapped expression profiles in a tabular format including FPKM, TPM, or even raw counts of transcripts and protein peptides are compatible with the ALAN pipeline. Therefore, ALAN users will not require raw sequencing files such as BAM or FASTQ, which may not be readily available. ALAN yields user-friendly intermediate and final output matrix files in tabular formats for users to subsequently review, interpret, and illustrate key findings with full customizability. Galaxy is one workflow manager in which users can identify gene-profiles, druggable targets, and relevant mutations in cancer. ALAN can be incorporated into current Galaxy workflows after the generation of mapped expression profiles. The pairing of a traditional workflow that both analyzes raw sequencing data and consolidates relevant cancer information with ALAN's networking approach can ultimately deepen our understanding of how context-specific gene behavior can be applied to cancer treatment. As described herein, ALAN outputs can use a conventional pathway enrichment tool, GSEA. While ALAN outputs agreed with GSEA findings, GSEA pathways are prefixed static definitions, and thus not specific to all ecosystems. As an example, upon comparing signaling as functions of prostate cancer development, some gene networks are consistently similar (i.e. AR and FOXA1) whereas others are highly dependent on the ecosystem (i.e. MYC). In situations where a disease biology is poorly understood, ALAN may be used to develop custom gene signatures for biomarkers that have critical clinical functions within that disease context. In this manner, ALAN may be leveraged as a diagnostic tool, utilized to identify one or more diagnostic tools, or even to determine treatment plans (e.g., identify one or more treatments or remove one or more treatments from a current treatment plan). Treatment plans may include treatments such as surgical removal of tissue, administration of one or more foods, vitamins, minerals, and/or pharmaceuticals, etc.

ALAN can identify unique gene signatures that are context specific which can be integrated into current AI/ML approaches or used to generate gene panels. Neural-networking algorithms, such as PNET₁₅, can leverage cancer genomic data to predict critical “classifiers” that are potential biomarkers or even precision targets. A neural-networking approach is built upon architectural features (inputs, outputs, connectivity) designed by the user. When using PNET to study mCRPC, the architecture consisted of a multi-layered hierarchical network structure of genes where their interaction was inferred by 3007 curated biological pathways. Rather than relying on preconceived pathways, ALAN can adaptively construct the gene networks directly from the input data by identifying systematic changes in the behavior of one gene (i.e. AR and MYC) in distinct ecosystems. The approach ALAN utilizes is distinct from computing the differences of gene expression profiles that classify multiple states. This allows for improved interpretation of each gene function based solely on the ecosystems of interest (i.e. prostate tissue, mCRPC, breast cancer subtypes). For these reasons, ALAN can construct the hidden layers embedded into machine learning tools such as PNET, particularly when the objectives are to associate clinical outcomes with genomic features in one specific type of cancer. Embedding the ALAN mCRPC ecosystems as the PNET layers may robustly enhance the discovery power and clinical utility. In this manner, a machine learning system may be constructed using ALAN components as at least some layers, such as hidden layers, in the machine learning system. As described herein, a machine learning system may thus be developed in which training biases are reduced due to the reliance on actual subject data, such as a matrix of molecular abundance values for each sample in the matrix.

Broadly, using ALAN to study gene networks may deepen understanding of how genes behave in distinct ecosystems including different subtypes and prior exposure to therapeutics. In the molecular subtyping of breast cancer, expression-based approaches are applied to examine genes involving hormone sensitivity. Through observation of ESR1, ESR2, and PGR in FIG. 3 using ALAN, each gene has a distinct networking pattern across all subtypes of breast cancer. Given that positive expression of these genes is associated with more than one subtype, the ALAN result suggests that absolute expression patterns are not always indicative of the same phenomenon. Further investigation into how these differential gene networking patterns contribute to overall mechanisms within specific subtypes of breast cancer could enhance our understanding of subtype specific gene behavior. Particularly, several RNA-based gene signatures have been developed as diagnostic or prognostic biomarkers to stratify prostate cancer patient outcomes. In tumor samples, these signatures inform the degree of AR activity (NEPC, AR Nelson), enzalutamide resistance, or overall disease risk. However, the gene list used in signatures were curated through recurrent laboratory findings or regression-based models. While they are without doubt co-expressed genes in specific settings, there may be limited understanding of the behavior of each gene within signatures. In addition, by virtue of applying the signature, the assumption that interrelationships of the genes within each signature can be adapted to persist in all contexts. The utilities of ALAN allow rapid modeling of gene programs that naturally occur in distinct contexts based on data that often already exists. This expands the development of novel, yet context-specific signatures that may have improved predictive power for therapeutic response, survival, or other outcomes.

Investigating gene networks using ALAN networks may improve detection of signal activation of genes in distinct ecosystems. This can be used for purposing or repurposing cancer therapeutics and enhances investigations of therapeutic sensitivity and patient prognosis. In biomarker studies, BRCA1/2 dysregulations are used to stratify patients for the use of PARP inhibitors, but a subset of patients do not respond to this targeted treatment. One can examine differential interactions within ALAN BRCA1/2 networks in the ecosystems that consist of either responsive or non-responsive patients. In studying tumorigenesis and patient prognosis, users can leverage ALAN to study why losses or mutations in genes like P53 contribute to tumorigenesis or poor survival in a cancer type or tissue specific manner. Since ALAN allows for the investigation of the same gene in multiple ecosystems, there is also value in conducting analyses on other genes that are not oncogenes or tumor suppressors but have pleiotropic phenotypes, including chromatin and epigenomic modifiers, transcription factors, and immunoglobulins. ALAN can thus be used as a computational tool that both creates and interprets gene networks in the context of gene ecosystems while maintaining the input data architecture. Given the data, ALAN can be leveraged to aid development of precision biomarkers and to purpose novel or current therapeutics to additional patient populations.

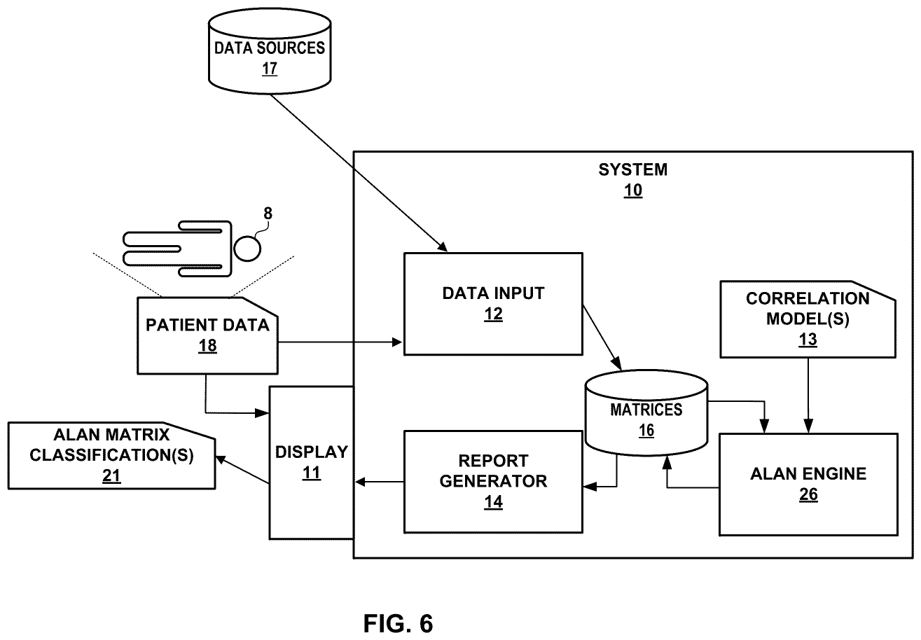

FIG. 6 is a block diagram illustrating an example computing system 10 for generating ALAN gene profiles. As detailed below with respect to FIG. 6, system 10 may represent a computing device or computing system, such as a mobile computing device (e.g., a smartphone, a tablet computer, a personal digital assistant, and the like), a desktop computing device, a server system, a distributed computing system (e.g., a “cloud” computing system), or any other device capable of receiving patient data 18 and performing the techniques described herein.

As further described herein, system 10 implements ALAN engine 26 to generate various metrics related to gene expression from sample data, such as gene relationships, and gene ecosystems. ALAN engine 26 may utilize one or more correlation models 13 to generate additional matrices from an initial matrix of molecular abundance values associated with one or more genes from each sample. The samples may be collected from one or more patient 8 via patient data 18 and/or sample data from other patients via other data sources 17. All of this sample data can be collected as data input 12, which can be arranged in an initial matrix by system 10 and stored as one of matrices 16. This initial matrix may include raw molecular abundance values for each sample. Matrices 16 may also include, once generated, the additional matrices generated by ALAN engine 26 using the one or more correlation models 13. For example, as descried herein, ALAN engine 26 may generate a first matrix by applying a first statistical correlation model to the initial matrix of raw data, and then generate a second matrix by applying the second statistical correlation model to the first matrix.

System 10 may then generate visualization or any other output via report generator 14 from one or more matrix stored in matrices 16. For example, report generator 14 may generate any metric corresponding to different correlations from the genes of the initial matrix as determined from the first or second matrix generated using the statistical correlation models. This output data can be sent to a display, such as display 11, and presented as one or more ALAN matrix classifications 21. These ALAN matrix classifications 21 include one or more of any of the graphs, charts, or data sets described herein, as some examples. In some examples, ALAN matrix classifications 21 may include diagnoses for one or more patients, classification of one or more tissues associated with one or more samples, genes related to various known treatments, or even a treatment plan or an adjustment to a treatment plan based on gene signatures, gene correlations, or any other information obtained from the correlations described herein.

As described herein, system 10 may include a data repository, such as matrices 16, configured to store molecular abundance values for a plurality of samples. System 10 may then include processing circuitry configured to receive, from the data repository, one or more matrices comprising at least one molecular abundance value for each sample of the plurality of samples, generate a first correlation matrix by at least performing a first statistical correlation operation between every pair of columns within the one or more matrices, generate a second correlation matrix by at least performing a second statistical correlation operation between every pair of columns within the first correlation matrix, determine, based on the second correlation matrix, a metric indicative of gene expression for one or more tissue types of at least one sample of the plurality of samples, and output, for display, the metric indicative of the gene expression. In this manner, the processing circuitry of system 10 may execute ALAN engine 26, for example, to perform these correlations and generate the matrices using correlation models 13.

The molecular abundance value may include at least one of a gene expression value, a gene count, or a protein abundance value. Other types of values may be used as well. The molecular abundance value may be representative of some measured value corresponding to a characteristic of gene expression for a particular gene in a respective sample.

In some examples, each row of each matrix of the one or more matrices of raw data includes molecular abundance values corresponding to one sample, and wherein different columns of each matrix of the one or more matrices corresponds to different types of molecular abundance values. The first method of the first statistical correlation may be different than a second method of the second statistical correlation. In one example, the first method comprises a rank-based Spearmans's correlation as one of correlation models 13, and the second method comprises a Pearson's correlation as another correlation model 13.

The first correlation matrix generated from the raw data may identify one or more gene profiles from the molecular abundance values of the one or more matrices. The second correlation matrix generated from the first correlation matrix may identify a gene ecosystem from the one or more gene profiles of the first correlation matrix.

In some examples, the processing circuitry is configured to determine a gene profile correlation between two gene profiles from individual cells of the second correlation matrix. The processing circuitry may be configured to determine a gene network correlation between two or more genes based on all rows of the second correlation matrix and two or more columns of the second correlation matrix corresponding to the two or more genes. In one example, the processing circuitry is configured to determine gene network signatures for any genes having correlation scores above 0.7 from the second correlation matrix. Then, the processing circuitry may be configured to determine a network score for a gene ecosystem from any gene network signatures for any genes having correlation scores above 0.7.

The matrices generated by ALAN engine 26 may provide information that is a practical use of the information within each matrix. For example, the processing circuitry may be configured to determine, based on a metric indicative of gene expression, a diagnosis of a condition associated with at least one sample of the plurality of samples. The metric indicative of gene expression may include gene correlations, gene profiles, gene networks, gene ecosystems, gene signature, gene ecosystem signature, or any other metric from which the processing circuitry can identify a diagnosis or related condition for a sample. In some examples, the processing circuitry may be configured to determine, based on the metric indicative of gene expression, a treatment plan for a condition associated with at least one sample of the plurality of samples. The treatment plan may include one or more pharmaceuticals or other interventions based on correlations, or non-correlations, to other genes associated with other conditions or treatments

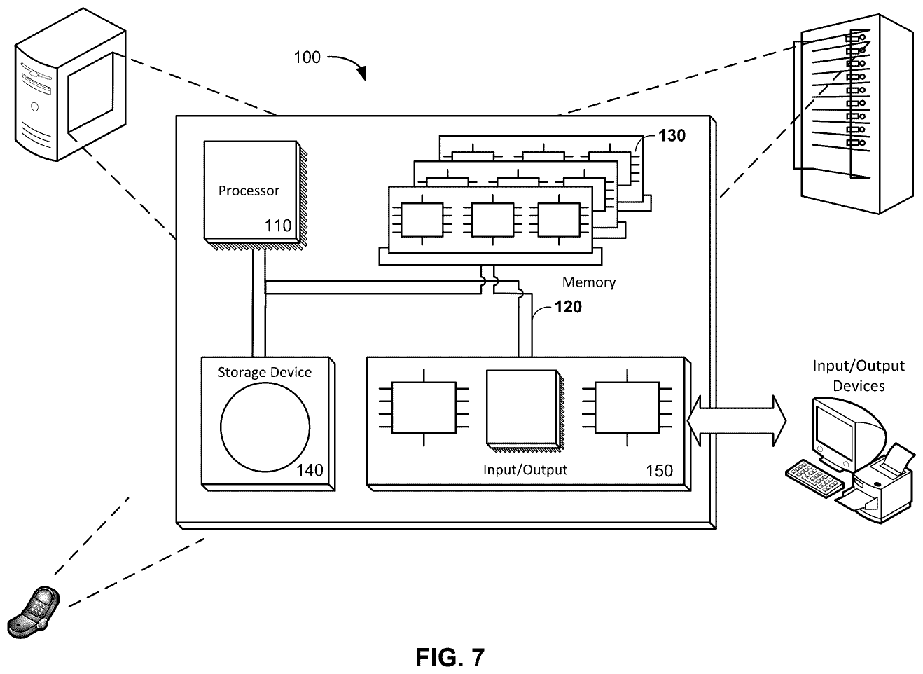

FIG. 7 is a block diagram illustrating various example devices that may be configured to implement one or more techniques of the present disclosure. That is, device 100 of FIG. 7 provides an example implementation of system 10 of FIG. 6 for determine gene correlations or other metrics corresponding to gene expression in one or more samples. Computing device 100 may be a mobile device (e.g., a smartphone, laptop, tablet, a personal digital assistant [PDA], or other mobile device), a workstation, a computing center, a cluster of servers, or other examples of a computing environment, centrally located or distributed, that is capable of executing the techniques described herein. Any or all of the devices may, for example, implement portions of the techniques described herein for the ALAN system. In some examples, ALAN functionality from system 10 may be distributed across multiple computing devices, such as a cloud-based computing system for computing the predicted scores and generating the reports, and a client device, such as a tablet or mobile phone, for accessing and viewing the reports.

In the example of FIG. 7, computing device 100 includes a processor 110 that is operable to execute program instructions or software, causing the computer to perform various methods or tasks, such as performing the techniques for generating ALAN outputs as described herein. Processor 110 is coupled via bus 120 to a memory 130, which is used to store information such as program instructions and/or other data while the computer is in operation. A storage device 140, such as a hard disk drive, nonvolatile memory, or other non-transient storage device stores information such as program instructions, data files of the multidimensional data and the reduced data set, and other information. The computer also includes various input-output elements 150, including parallel or serial ports, USB, Firewire or IEEE 1394, Ethernet, and other such ports to connect the computer to external devices such a printer, video camera, display device, medical imaging device, surveillance equipment, or the like. Other input-output elements include wireless communication interfaces such as Bluetooth, Wi-Fi, and cellular data networks.

Computing device 100 may itself be a traditional personal computer, a rack-mount or business computer or server, or any other type of computerized system. Computing device 100, in a further example, may include fewer than all elements listed above, such as a thin client or mobile device having only some of the shown elements. In another example, computing device 100 is distributed among multiple computer systems, such as a distributed server that has many computers working together to provide various functions.

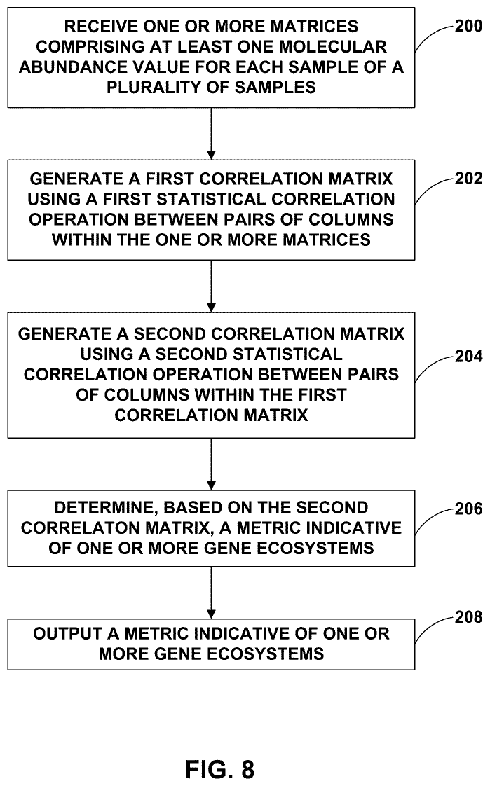

FIG. 8 is a flow diagram illustrating an example technique for generating an ALAN gene profile. The example of FIG. 8 is described with respect to system 10 and ALAN engine 26 executed by processing circuitry of system 10, but may be executed by other components of system 10 or other systems, such as processor 110 of system 100.

As shown in the example of FIG. 8, ALAN engine 26 may receive one or more matrices comprising at least one molecular abundance value for each sample of a plurality of samples (200). ALAN engine 26 then generates a first correlation matrix using a first statistical correlation operation between pairs of columns within the one or more matrices (202). Next, ALAN engine 26 can generate a second correlation matrix using a second statistical correlation operation between pairs of columns within the first correlation matrix (204).

ALAN engine 26 can, based on the second correlation matrix, a metric indicative of one or more gene ecosystems (206). In some examples, ALAN engine 26 can determine the metric from the first correlation matrix. In other examples, report generator 14 may generate the metric from one or more of the matrices generated from the raw data. ALAN engine 26 and/or report generator 14 may then output the metric indicative of one or more gene ecosystems (208). As described herein, the one or more gene ecosystems may thus be determined from molecular abundance values, such as gene expression values from different samples.

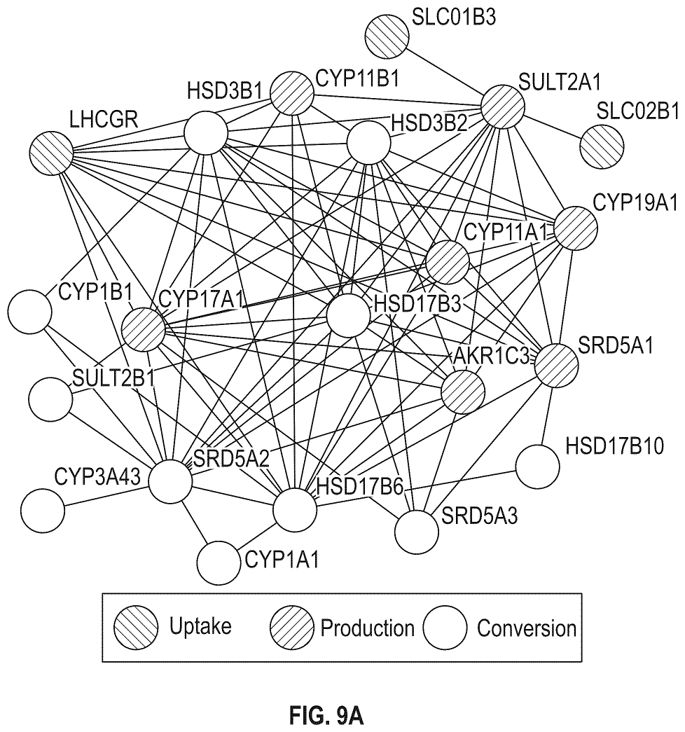

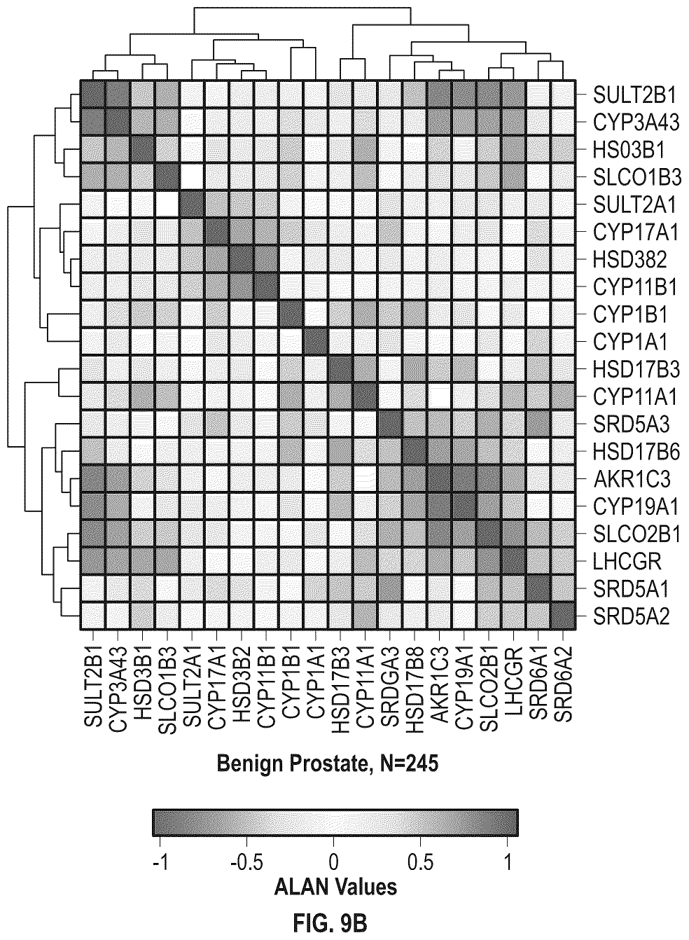

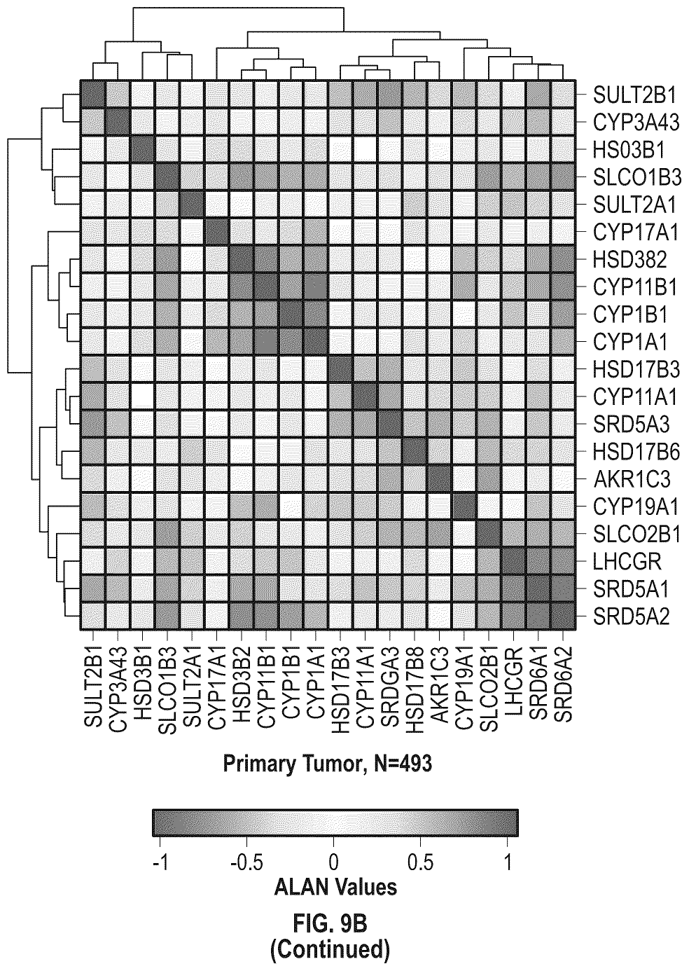

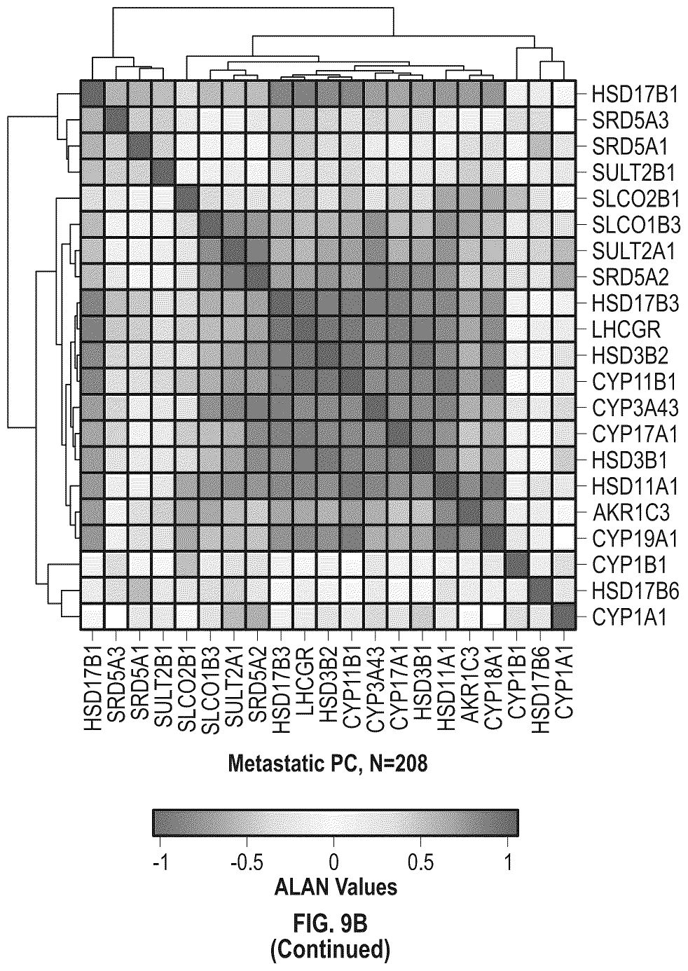

FIGS. 9A-23B relate to an example of implementing ALAN for determining relationships of genes, such as gene ecosystems, for prostate cancer. Prostate cancer (PC) is driven by aberrant signaling of the Androgen Receptor (AR) and patients with PC are managed by androgen deprivation therapies (ADT). ADT responsiveness is associated with germline alterations in genes that regulate androgen production, uptake, and conversion (APUC). As described below and with respect to FIGS. 9A-23B, a system was used to computationally examine the intratumoral RNA expression of 21 known APUC genes from prostate tumors and evaluated their relationship with clinical outcomes. Six APUC genes (HSD3B1, HSD3B2, CYP3A43, CYP11A1, CYP11B1, CYP17A1) exhibited coalescent gene behavior in a pilot cohort of metastatic tumors (n=208). In a confirmatory Caris dataset, the 6 APUC genes (APUC-6) exhibited robust clustering in prostate (n=4,490) and metastatic (n=2,593) biopsies. Surprisingly, tumors with elevated APUC-6 expression had statically lower expression of AR, AR-V7, and AR signaling scores. APUC-6 genes instead associated with the expression of steroid hormone receptors, ESR1/2 and PGR. In a confirmatory cohort, RNA expression of AR or APUC-6 genes were used to define two subgroups of tumors with differential association with hallmark pathways and cell surface targets. This also defined patients with opposing outcomes, in which APUC-6 high tumors had improved overall survival. Altogether, measuring the aggregate expression of APUC-6 genes in current genomic tests identifies PCs that require distinct therapeutic strategies. In this manner, a system can apply ALAN to gene expression for various genes in order to identify gene ecosystems that may be related to these distinct therapeutic strategies designed to address conditions or traits associated with one or more of the identified gene ecosystems.

In advanced prostate cancer, persistent activity of the Androgen receptor (AR) is a key driver of tumor progression, patient survival, and metastases. Numerous aberrancies leading to the retained signaling of AR have been described including non-hormonal drivers, promiscuity of other steroid hormone receptors (SHRs), amplification or mutation in the receptor itself, and, to a lesser extent, variation in the levels of ligands that lead to AR activation. In most cases, death from prostate cancer is marked by retained and persistent AR activity. Therefore, further exploration of novel inputs to its activity may be helpful.

Understanding the relationship between measurable androgens and patient outcomes is nascent. Previous studies have found positive associations between higher serum levels of androgens and patient outcomes in settings in which androgen-lowering drugs are subsequently deployed. Opposed to serum levels, intratumoral androgen production is a driver of tumor progression, and our understanding of its relationship to tumor status and patient outcomes is incomplete. Androgens measurable in the serum typically reflect an adrenal source, whereas the technical feasibility of quantifying tumor-produced androgens is currently not possible. Alternatively, the availability of intratumoral sequencing techniques now enables the study of such production.

Androgens may enter tumor cells directly or be produced within the tumor through conversion of precursors to androgen molecules. This occurs through an enzymatic process involving a suite of more than 20 genes that govern androgen production, uptake, and conversion (APUC). Given the essential need for these steroid molecules or ligands in driving tumor growth, variation in such genes may significantly impact outcomes in prostate cancer, either singularly or in aggregate. One critical APUC gene is CYP17A1, which encodes Cytochrome P450 17A1. This enzyme converts

pregnenolone and progesterone to 17-OH Pregnenolone and 17-OH progesterone and 17-OH-pregnenolone to DHEA primarily in the adrenal glands. Such steroid intermediates are critical precursors of testosterone (T) and dihydrotestosterone (DHT). T and DHT are extremely potent activators of AR which led to the pharmacologic development of abiraterone. Insights into androgens of adrenal 7 and/or intratumoral origin 6 drove the clinical development of the androgen synthesis inhibitor abiraterone acetate. This therapy may be a standard-of-care treatment for patients with advanced PCs across many clinical states because it directly targets CYP17A1, preventing the generation of T and DHT. Beyond abiraterone, opevesostat, which inhibits CYP11A1, is the only other drug specifically designed to ablate the activity of an APUC gene that is available for patients with prostate cancer.

Germline genetic studies further support the examination of the clinical impact of APUC genes in prostate cancer. The APUC gene 3β-hydroxysteroid dehydrogenase-1 (encoded by HSD3B1) is primarily expressed in the peripheral, nonendocrine, tissues of the body and catalyzes the conversion of DHEA into androstenedione and other downstream androgens. The protein product of HSD3B1 regulates the production of non-testicular T or DHT12 which are high affinity ligands that activate AR. One variant of HSD3B1 in which sustained androgen synthesis is achieved through enzyme stabilization, is associated with resistance to AR-targeted therapies and subsequently unfavorable clinical outcomes. Beyond HSD3B1, variants of other APUC genes have been implicated in poor patient outcomes. This includes the gene encoding the organic ion transport proteins, SLCO2B118,19, CYP19A120, CYP3A421,22, and SRD5A223, which results in the conversion of T to DHT, creating the most potent AR agonist. Altogether, previous studies have demonstrated that germline genetic dysregulations in APUC genes can lead to poor patient outcomes which suggests that intratumoral alterations such as RNA levels measured by RNA-seq could also drive AR activation and therapy resistance.

The impact of intratumoral alterations in either DNA or RNA of APUC genes is not well characterized. With 21 potential APUC genes, a challenge for defining their relationship to prostate cancer is that many intratumoral changes are detected in advanced prostate tumors and they are often not mutually exclusive. To address this and similar challenges, a system can employ the ALAN as a computational tool configured to interpret gene networks within transcriptomic patient data by quantifying the relationship between all genes of interest. Given the complexity of APUC genes, and their many possible interactions, the system can employ this unbiased approach of ALAN to determine the intratumoral relationship between APUC genes within large PC patient datasets. In this example, the process includes the analysis of the associations of 21 genes known as enzymatic regulators of androgen production, uptake, and conversion (APUC) 16 to determine their relationship prostate cancers.

FIG. 9A is a string analysis of degrees of connection indicative of interation between different genes in prostate tissue. The STRING analysis was performed to indicate the degree of connection of different genes. Based on the output for molecular interactions, the different types of genes are labeled uptake, production, and conversion, genes. According to the results, six APUC genes exhibit concordant behavior in metastatic PC. Unbiased approaches were used to study the interplay between the 21 APUC genes. The process used STRING technique to conduct an iterative search in public databases in order to assess the strength of pairwise gene interactions as shown in FIG. 9A. While this confirmed that the family of APUC genes is largely associated with one another, the process was unable to deconvolute which APUC gene interactions are relevant for the pathogenesis of PC. Next, the system applied ALAN to quantify all pairwise gene-to-gene interactions based on a continuous measurement (i.e. RNA expression), which was used to model the changes in AR interactions in benign prostate tissue, primary PC, and metastatic PC.

The system performed ALAN on the transcriptomes from samples annotated as benign prostate tissue (GTEx, n=245), primary PC (TCGA, n=493), and a pilot cohort of metastatic PCs (SU2C/PCF, n=208). Unsupervised clustering of ALAN outputs indicated that 6 out of 21 APUC genes, HSD3B1, HSD3B2, CYP11A1, CYP11B1, CYP17A1, CYP3A43, exhibited increasing association with respect to disease progression from benign to primary PC but only exhibited robust association in metastatic PC indicating context specific coalescent behavior, as shown in FIG. 9B. FIG. 9B are thus charts of example relationships between genes exampled using ALAN outputs from different tissue types. The relationship between APUC genes was examined using ALAN outputs, values between −1 and 1, based on WTS data from benign prostate tissue, PC, and metastatic PC tumors. Unsupervised hierarchical clustering was performed on ALAN outputs within each dataset. 6 APUC genes are highlighted.

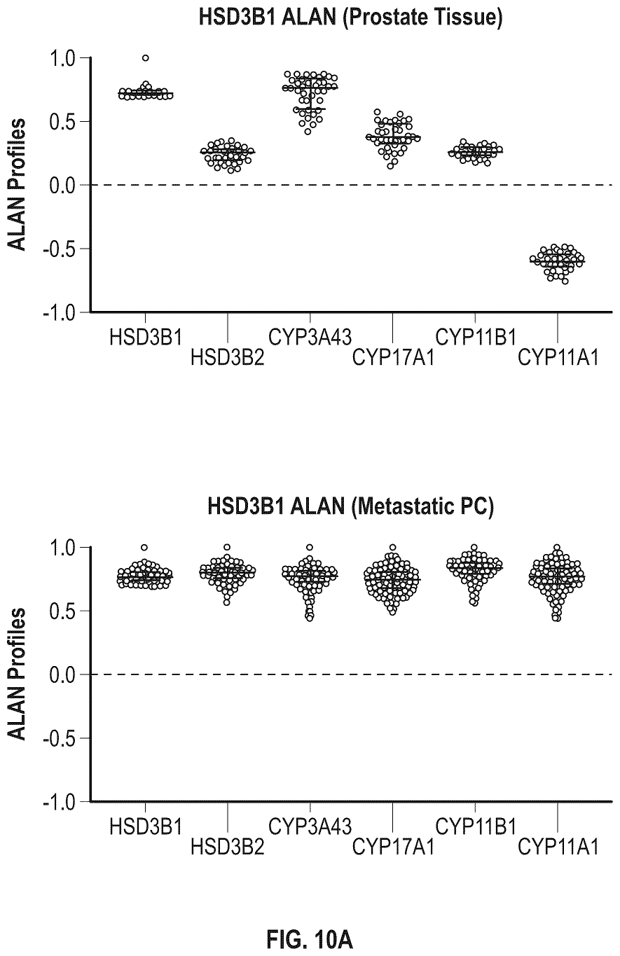

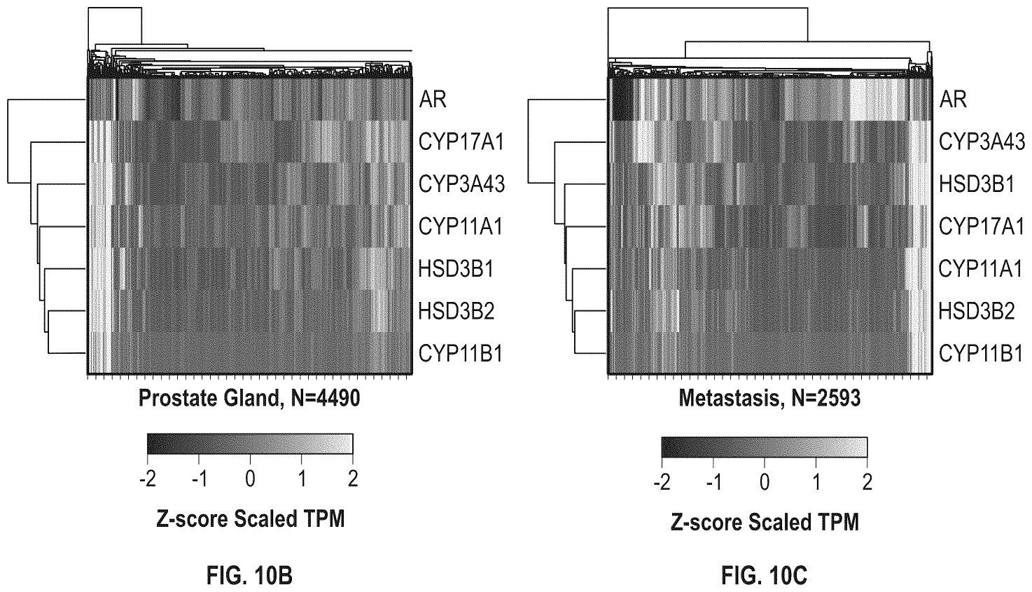

FIG. 10A is a graph of example ALAN profiles for example genes of interest for prostate tissue and metastatic prostate cancer tissue. The ALAN profiles for 6 APUC genes of interest are examined with greater detail in prostate tissue and metastatic PC. The six genes were examined in greater detail based on their ALAN profiles and again found they exhibited high concordance, but only in metastatic PC and not in benign prostate tissue, as shown in FIG. 10A.

FIGS. 10B and 10C are graphs of example hierarchical clustering of different tissue samples. Using WTS data from the Caris dataset, unsupervised hierarchical clustering of prostate and metastatic PC samples was conducted based on Z-score scaled TPM data. As shown in FIGS. 10B and 10C, unsupervised clustering of prostate tumor biopsies (Caris, n=4,490) and metastatic PC biopsies (Caris, n=2,593) indicated that the expression of the six APUC genes was again clustered as evident by the origin at the same branch point, but interestingly not with AR as it is the furthest branch point from APUC genes.

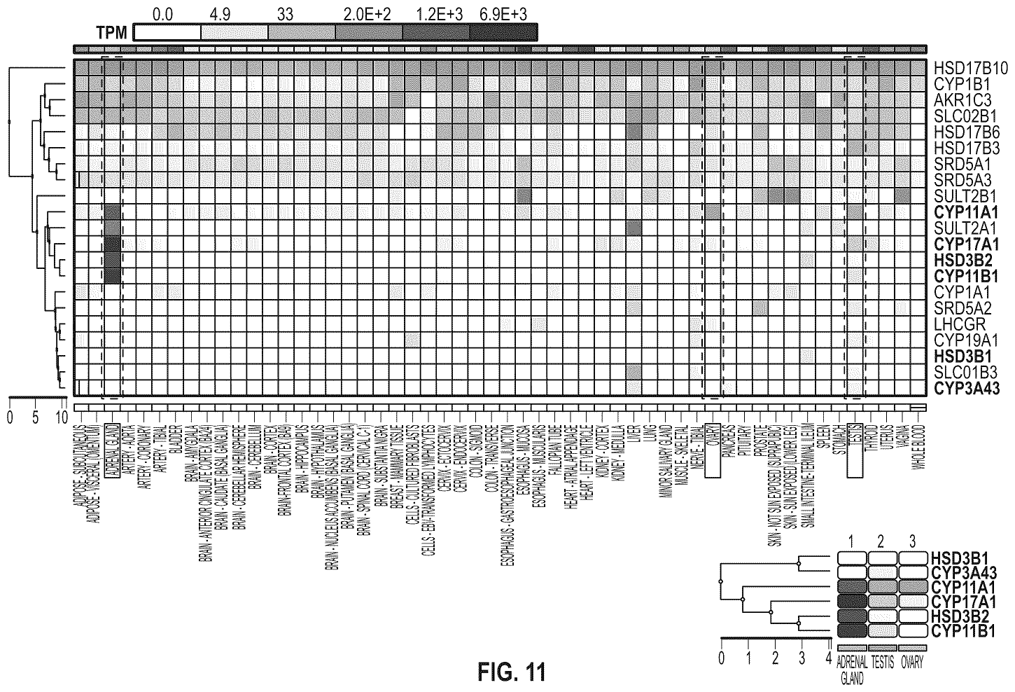

FIG. 11 is an example graph of expression of example genes across a plurality of tissue sites. The median expression (TPM) of all APUC genes were examined in the GTEx database across all available tissue sites. 6 APUC genes are highlighted. When the expression of APUC genes was examined in RNA-seq data from benign tissue (GTEx), at least one of the six APUC genes exhibited high expression in the adrenal gland, testis, and ovary (top three expressed tissues of 6 APUC genes, as shown in FIG. 11). Altogether, HSD3B1, HSD3B2, CYP11A1, CYP11B1, CYP17A1, CYP3A43, hereby defined as APUC-6, demonstrated tissue—and cancer stage—specific interactions, with the most notable interaction in metastatic PC. APUC-6 genes define a subset of metastatic PCs with reduced AR activity. As the APUC-6 genes did not exhibit the expected association with AR, the process further examined the AR activity in tumors with elevated APUC-6 expression.

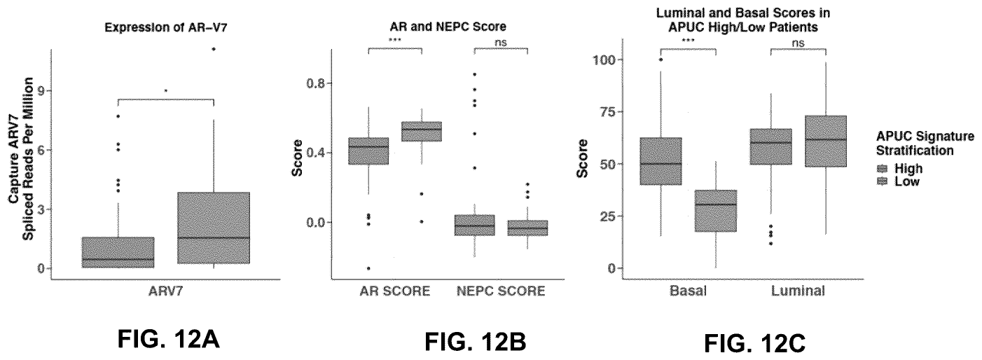

FIGS. 12A, 12B, and 12C are bar graphs of different expression and scores of example genes. In the SU2C/PCF study, metastatic PCs with elevated APUC-6 expression had reduced AR-V7 expression (FIG. 12A) and AR activity (FIG. 12B). These tumors were not associated with increased NEPC scores and exhibited elevated basal—but not luminal-like profiles based on previously defined single cell RNA-seq signatures 28 (FIG. 12C). APUC-6 genes were used to stratify metastatic PC patients from the SU2C/PCF samples in which were examined in FIG. 12A. The relative expression of AR-V7 (p-value), FIG. 12B. AR and NEPC signatures (adjusted p-values) as shown in FIG. 12C. Luminal and Basal signatures (adjusted p-values). P-values for single tests or adj p-values for multiple comparisons are shown (adj/p-value >0.05 (ns), adj/p-value <=0.05 & >0.01 (*), adj/p-value <0.01 & p-value >=0.001 (**), adj/p-value <=0.001 (***)).

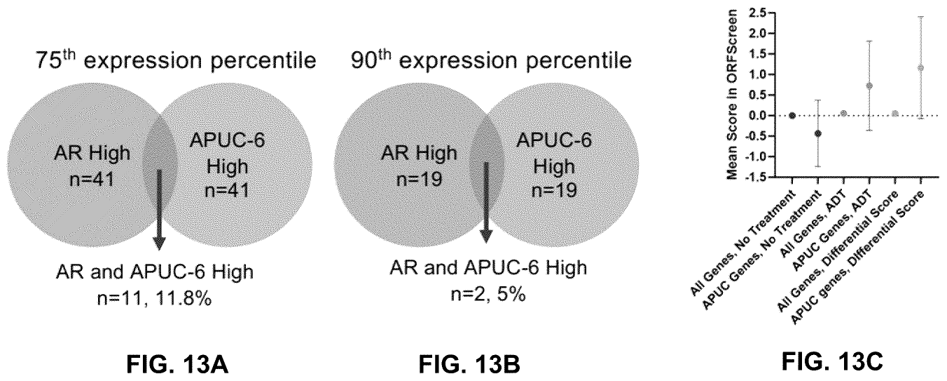

FIGS. 13A and 13B are Venn diagrams of expression for different cancers at different expression percentiles. Venn diagrams showing co-expression of AR high and APUC-6 high prostate cancers (SU2C/PCF) using two percentile thresholds—above

75th percentile and 90th percentile of target gene(s) expression. To establish which patients exhibited high AR expression or high APUC-6 expression, the process determined the number of patients with high AR expression (>75th, >90th percentile; n=52, n=21) and high APUC-6 expression (>75th, >90th percentile; n=52, n=21) in the SUC2/PCF dataset. At the 75th percentile threshold (FIG. 13A), 11.8% (n=11) of patients co-expressed these signatures, while only 5% (n=2) co-expressed these signatures using a 90th percentile threshold (FIG. 13B).

FIG. 13C is a graph of example proliferation scores for example APUC genes compared to other genes in an overexpression screen. The proliferation score for the 6 APUC genes was aggregated as compared to the 17,249 other genes in an overexpression screen. The aggregate scores of genes based on 2 treatment conditions are then presented (No Treatment, ADT), as well as the differences in the proliferation scores for every

gene (Differential Score). The functionality of the APUC-6 genes in cells was evaluated based on a prior overexpression screen in AR-dependent LNCaP cell lines that included 17,255 genes. As compared to the 17,249 other genes, the aggregate viability score of APUC-6 genes were distinct (FIG. 13C), but the pro-proliferation effects were only observed when the cells were cultured in conditions that mimicked ADT (no steroid hormones and treated with enzalutamide). These results support the functional relevance of APUC-6 genes in the setting of ADT treatment; however, APUC-6 genes were surprisingly associated with reduced AR-V7 and AR activity.

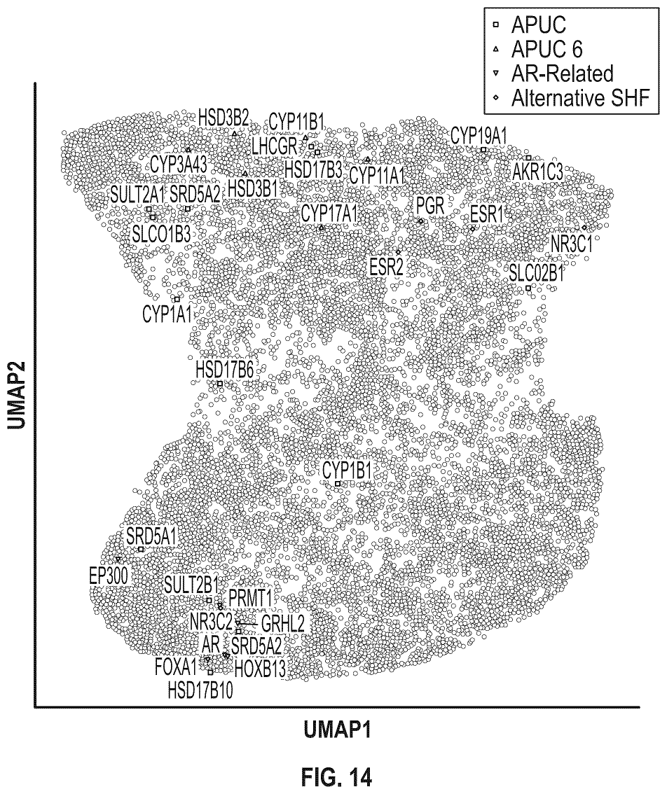

FIG. 14 is a map of distances between different genes according to ALAN gene behavior. UMAP was used for dimensional reduction of the ALAN outputs from metastatic PC patients, in which the distance between genes (grey dots) indicates the similarity of ALAN gene behavior. 4 groups of genes are specifically labeled in FIG. 14 as APUC (purple), APUC-6 (red), AR-Related (blue) 42, and Alternative SHRs (green). APUC-6 genes exhibit robust associations with ESR1, ESR2, and PGR in prostate and metastatic biopsies. In order to identify the relative interaction of APUC-6 genes with respect to all detectable genes in metastatic PC, the dimensional reduction of ALAN outputs is shown in FIG. 14. This approach enables visualization (e.g., via a user interface controlled by the system) of which genes behaved similarly to the APUC-6 genes. Here, APUC-6 genes were indeed in close proximity on the UMAP but were distant from AR and AR co-factors including HOXB13, FOX1, GRHL2, PRMT1, and EP300.

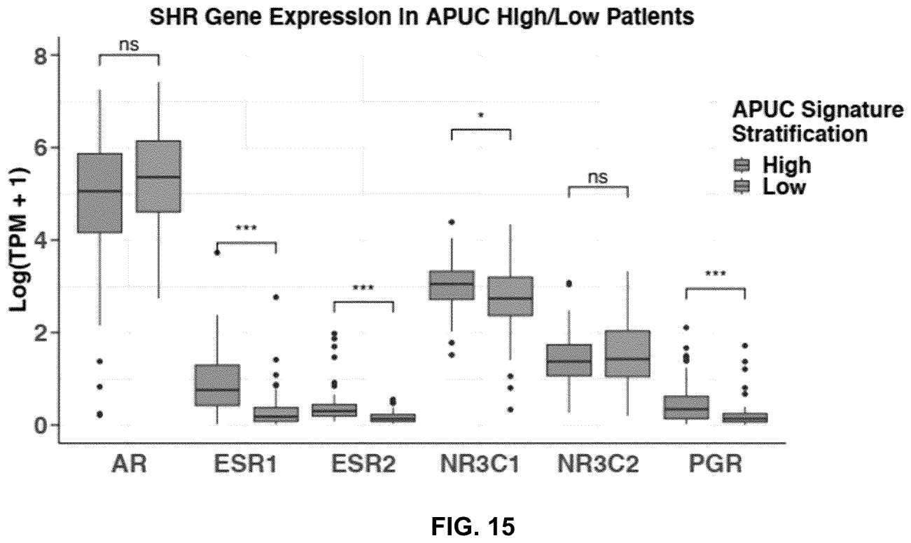

FIG. 15 is a bar graph of relative expression of cancer related hormone receptors. Based on stratifying patients by APUC-6 expression, we examined the relative expression of cancer related hormone receptors. Surprisingly, when examining all alternative steroid hormone receptors including ESR1, ESR2, PGR, NR3C1 (glucocorticoid receptor-GR), NR3C2 (mineralocorticoid receptor-MR), APUC-6 genes exhibited the most similarity to ESR1, ESR2, and PGR. Interestingly, ESR1 is a current therapeutic target in breast cancers. When revisiting the metastatic tumors stratified by APUC-6 levels, tumors with high APUC-6 expression had increased expression of ESR1, ESR2, and PGR but reduced expression of AR as shown in FIG. 15.

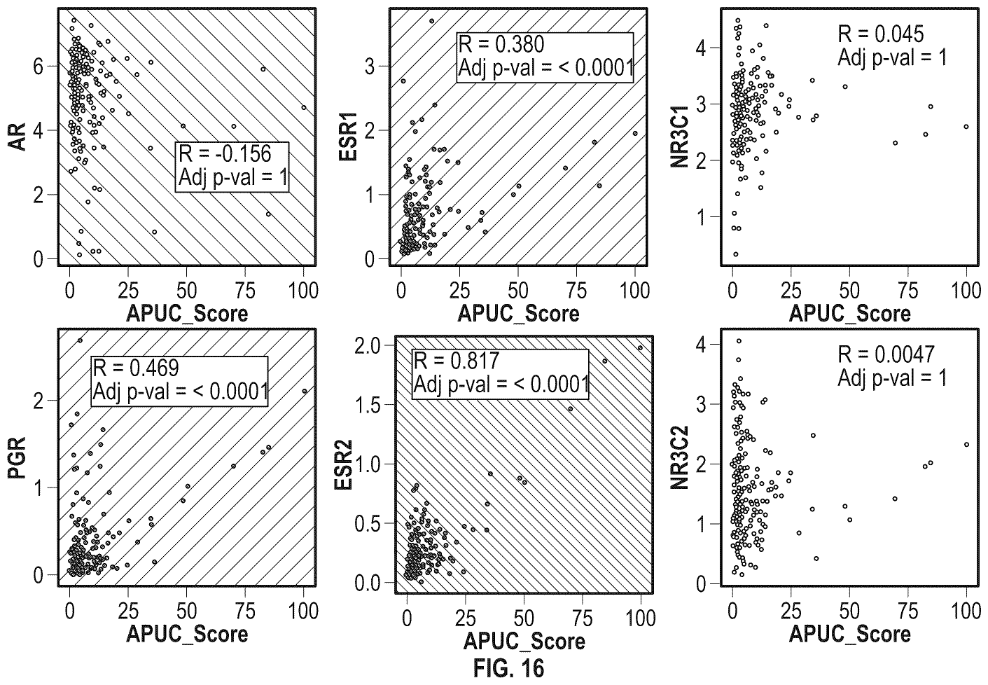

FIG. 16 includes Pearson correlations of relative expression of hormone receptors to the APUC genes. A Pearson correlation was used to examine the relative expression of hormone receptors with respect to APUC-6 genes (APUC-Score) in metastatic

PC samples. Using APUC-6 genes as a signature, we determined that the associations with ESR1, ESR2, and PGR were positive and significant (Adj. p-val <0.0001) while the association with AR, GR, and MR were not significant (FIG. 3C). The correlation coefficient (R) and adjusted p-values (FDR adjusted for multiple comparisons) are shown (adj. p-value >0.05 (ns), adj p-value <=0.05 & >0.01 (*), adj p-value <0.01 & pval >=0.001 (**), adj p-value <=0.001 (***)).

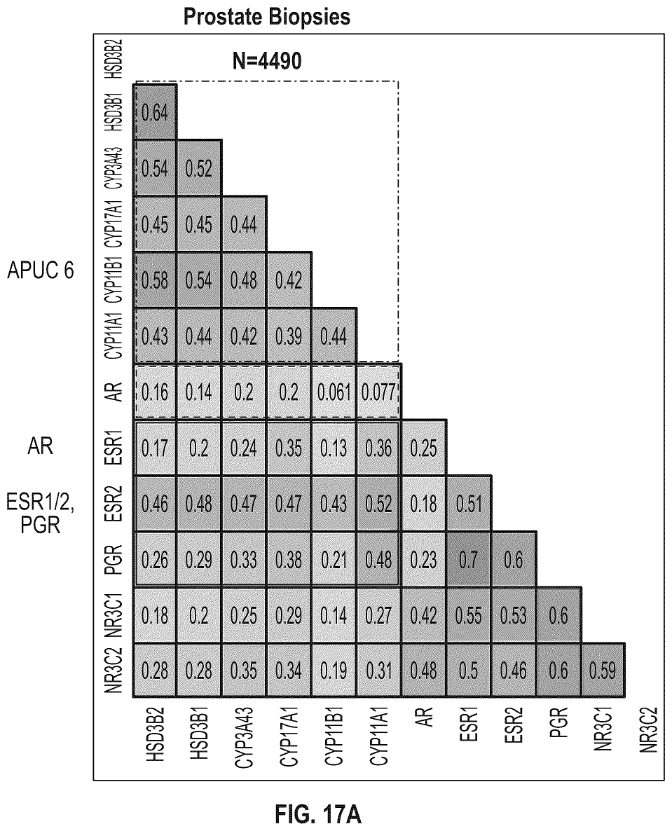

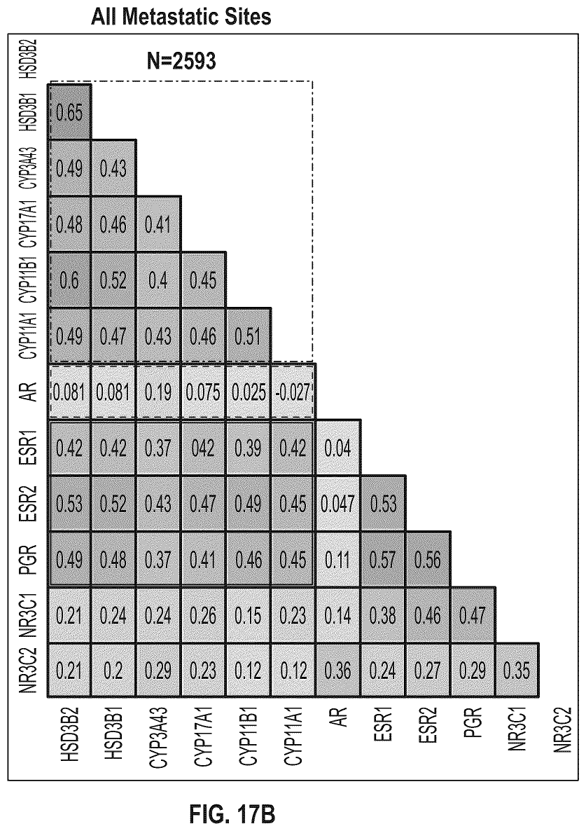

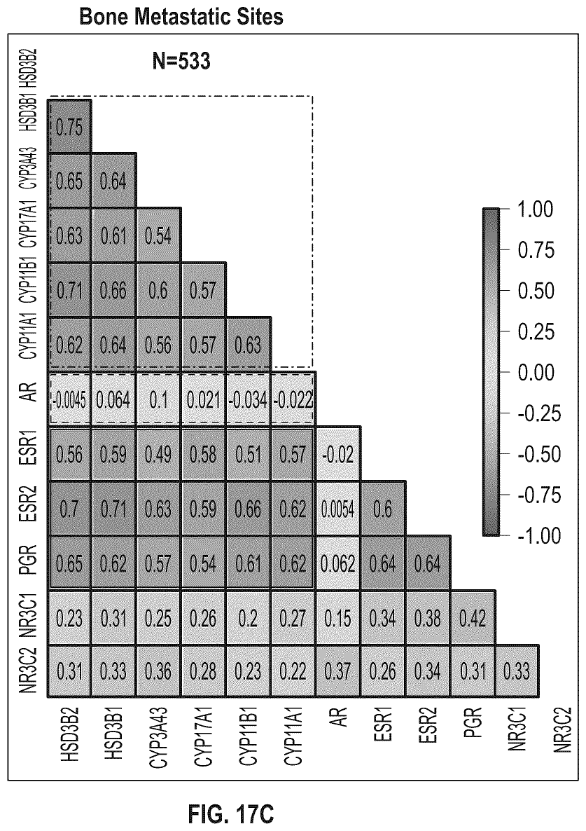

FIGS. 17A, 17B, and 17C are correlation matrices illustrating Spearman correlations between APUC genes and hormone receptors. The relationship between APUC-6 genes with the expression of all hormone receptors in the larger Caris dataset were determined. To do so, the expression of each APUC-6 gene was cross correlated with the expression of all other steroid hormone receptors. In the 4,490 prostate tumor biopsies, APUC-6 genes exhibited a robust correlation with one another, and all had strong positive correlations with ESR1, ESR2, and PGR (Spearman 0.13-0.52), while the correlation with AR was relatively weak (Spearman 0.06-0.20). In the 2,593 metastatic samples, we observed similar associations among the APUC-6 genes, but with greater correlation with ESR1, ESR2, and PGR (Spearman 0.37-0.53), while the association with AR was further diminished (Spearman-0.03-0.19). Indeed, as shown in the examples of FIGS. 17A, 17B, and 17C, APUC-6 genes have positive associations with ESR1, ESR2, PGR, but not AR. In the Caris cohort of samples, the Spearman correlation between APUC-6 genes, AR, ESR1, ESR2, PGR, GR (NR3C1), and MR (NR3C2) were examined. The overall results are shown in FIGS. 17A-17C as a correlation matrix and separately examined tissue from prostate tumors, metastatic PCs, and PCs that metastasized to the bone.

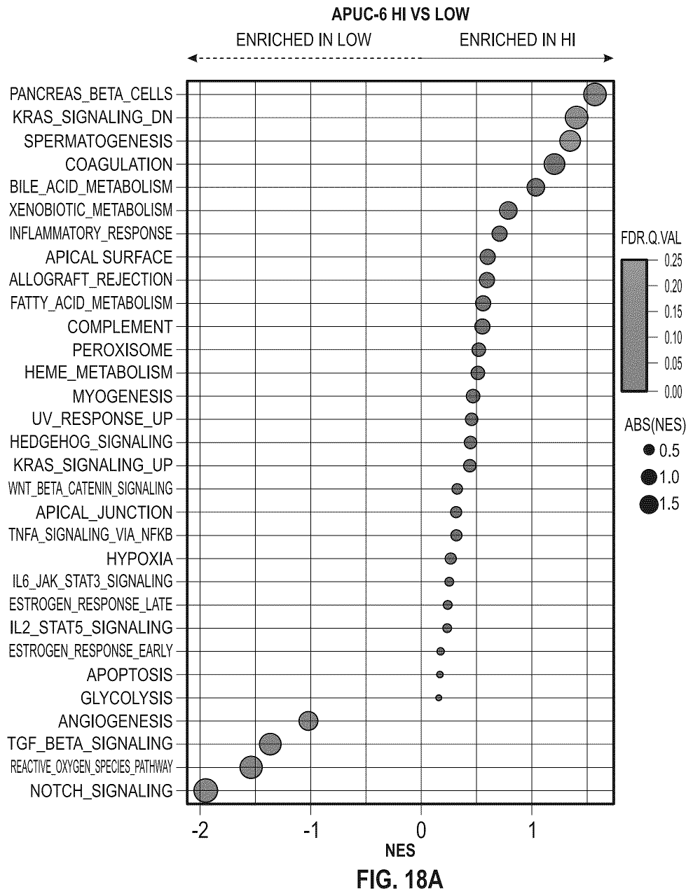

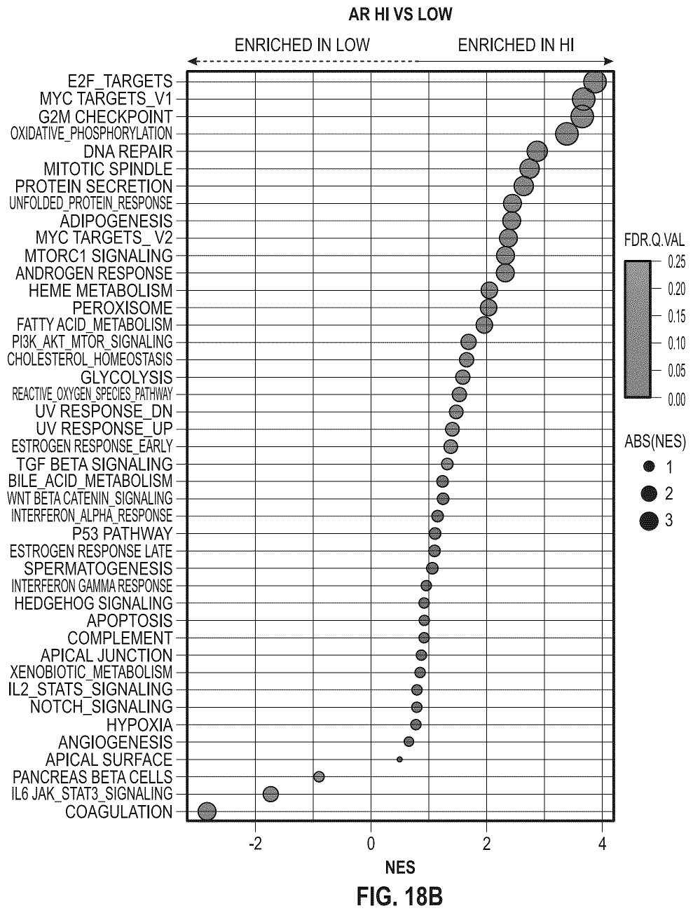

FIGS. 18A and 18B are graphs of example Hallmark signatures for different status of APUC genes. The same analysis was performed upon stratifying samples by metastatic biopsy sites including lymph node (n=833), bone (n=533), liver (n=360), bladder (n=313), lungs (n=114), brain (n=24), and the adrenal gland (n=22). APUC-6 genes exhibited robust correlation regardless of metastatic tissue sites, with the most robust association in the 22 adrenal gland samples. However, we also note that normal adrenal gland tissue also had the highest expression of APUC-6 genes. The three steroid hormone receptors ESR1, ESR2, and PGR exhibited the strongest correlation (Spearman 0.49-0.71) with APUC-6 genes in the 533 bone metastasis samples. Similar to what we found in the pilot SU2C/PCF cohort, APUC-6 genes exhibited the strongest correlation with ESR2 across all metastatic sites. The only tissue site in which APUC-6 genes had a notable AR correlation was in the adrenal gland, in which we also noted that APUC-6 genes are generally expressed in the benign prostate tissue. When patients were stratified based on self-reported race (European American, African Americas, Asian Pacific Islander), the degrees of associations showed consistent trends across populations (Supplementary FIG. 2). Altogether, APUC-6 genes exhibited consistent association with each other in prostate tumor biopsies and all metastatic tumor biopsies regardless of tissue sites. They generally had a positive association with ESR2, ESR1, and PGR across all metastatic sites and had limited to no association with AR. Pathway analysis indicates that APUC-6 genes exhibit divergent signaling as compared to AR. To examine the biological processes that are associated with AR or APUC-6 high tumors, we generated differential expression profiles and then examined the signaling pathways that are associated with these tumors using Gene Set Enrichment Analysis (GESA).

The pathway analysis of FIGS. 18A and 18B indicates APUC-6 high and AR high regulate distinct pathways. 50 Hallmark signatures were analyzed using GSEA based on APUC-6 high or AR high status. The analysis was conducted based on primary tumor samples from the Caris cohort. In prostate biopsies (Caris), we found that APUC-6 genes were associated with increases in Hallmark signatures including Pancreas Beta cells and KRAS signaling as shown in FIGS. 18A and 18B. Here, AR high tumors were expectedly associated with the Hallmark Androgen Response, MYC, and cell cycle (E2F) pathway.

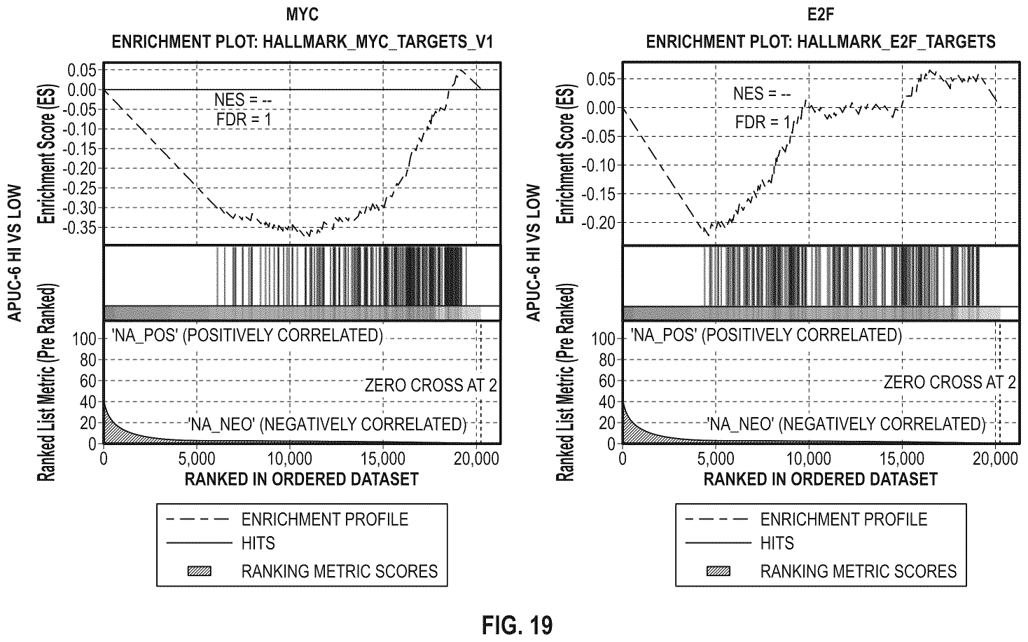

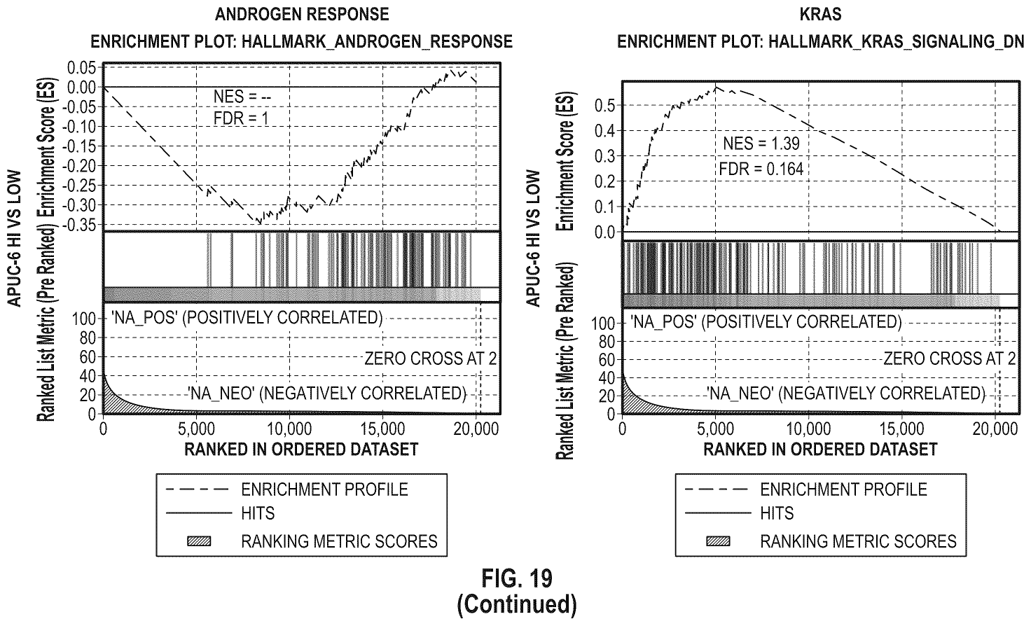

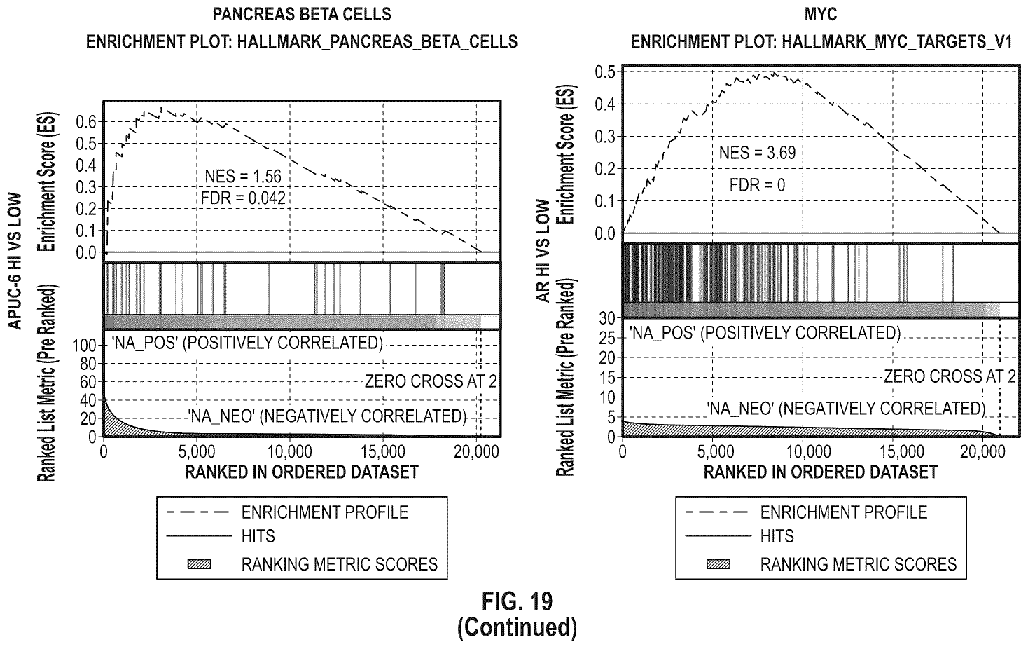

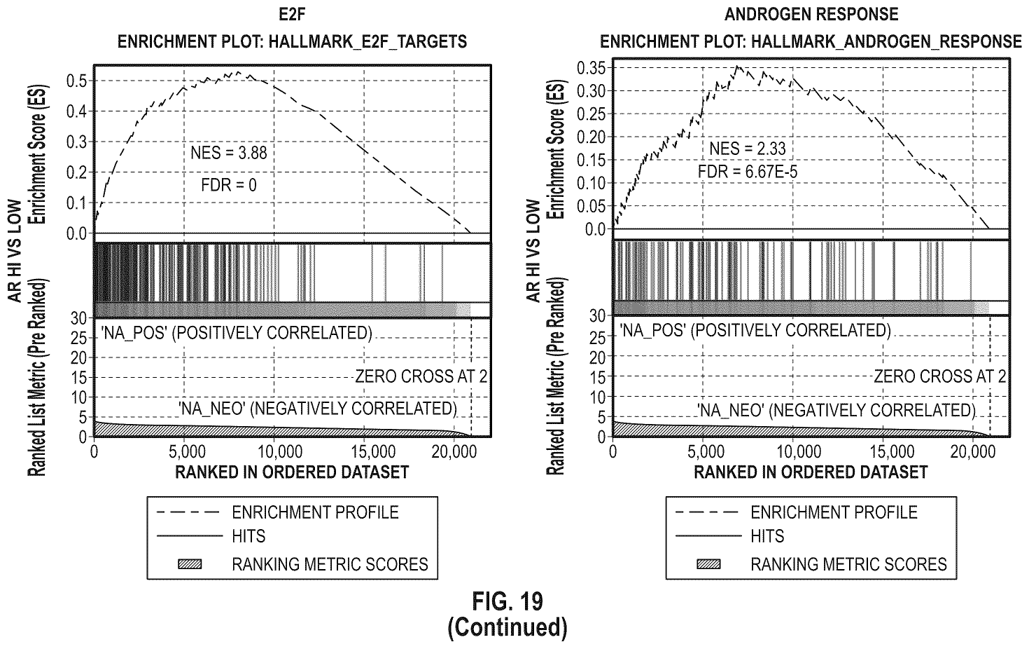

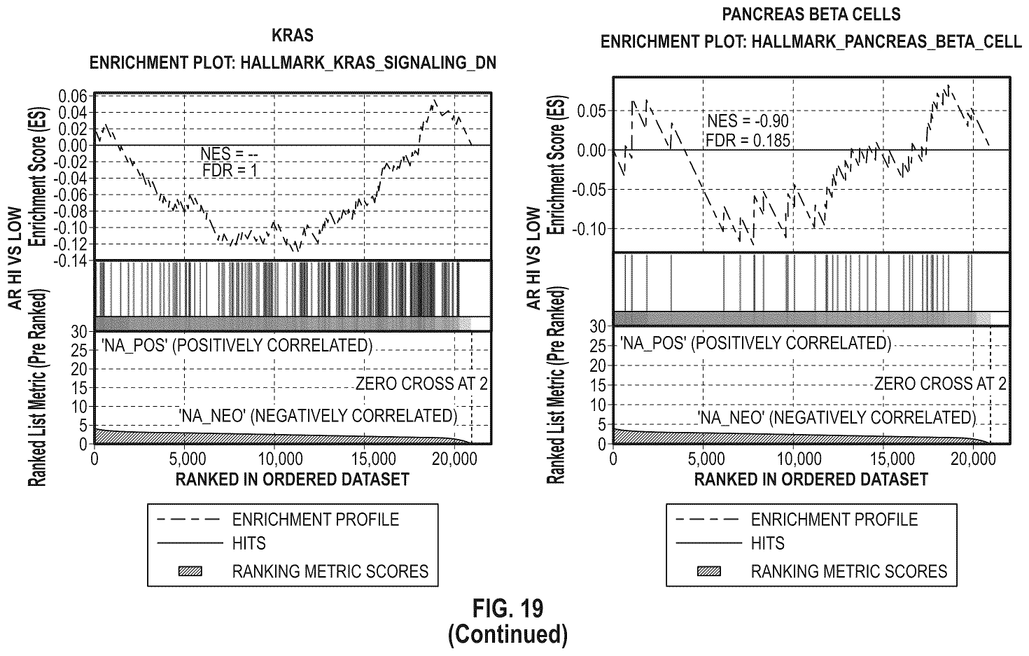

FIG. 19 illustrates plots indicating example enrichment scores and false discovery rates. The enrichment plots are shown along with net enrichment scores (NES) and false discovery rate (FDR). When we examined the GSEA enrichment plots for key pathways, including MYC, E2F, Androgen Response, KRAS, and Pancreas Beta cells, we noted that

the directionality of enrichment of these pathways was exactly opposite when comparing the APUC-6 high or AR high tumors (FIG. 19).

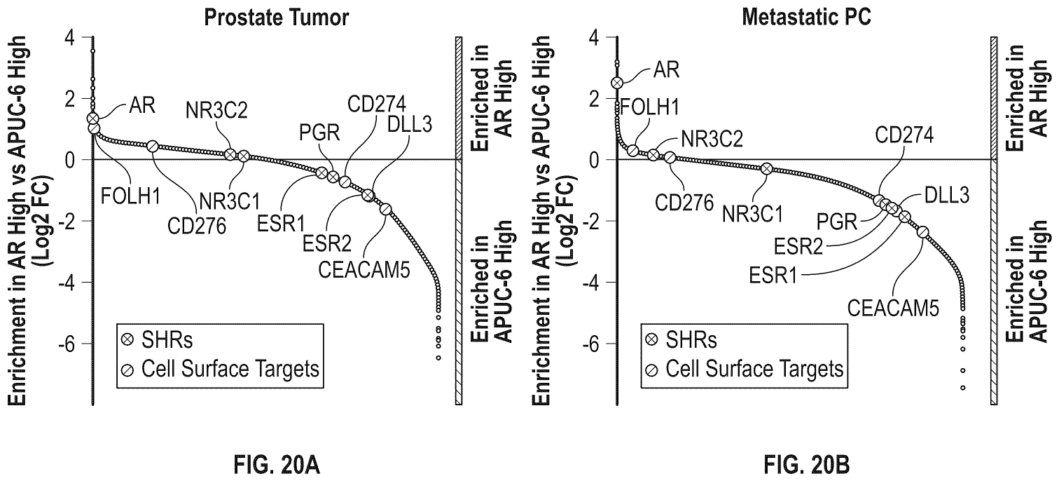

FIGS. 20A and 20B are graphs of relative rank of genes based on differential expression in different tissues. Next, the magnitude in which genes were differentially expressed in APUC-6 high or AR high tumors was examined to determine if these relationships differed between prostate biopsies and metastatic PC. Consistent

with the initial associations observed for the hormone receptors, APUC-6 genes were more associated with the expression of cell surface targets DLL3 and CEACAM5, as compared to PSMA (FOLH1) and B7-H3 (CD276) (FIGS. 20A and 20B). As shown in FIGS. 20A and 20B, the relative rank of all genes based on the differential expression upon grouping samples based on APUC-6 or AR expression status in primary tumor biopsies and metastatic tumor biopsies via snake plot. Genes enriched in AR High tumors have a positive enrichment score (gray) and APUC-6 high tumors have a negative enrichment score. Steroid hormone receptors (red) and cell surface targets (blue) are highlighted.

This remains true in both the prostate tumor and metastatic PC setting. As with the signaling pathways, AR high tumors essentially had reciprocal relationships with the expression of each of these genes. While the interrogation of the transcriptomes does not fully explain the mechanisms that distinguish tumors with high APUC-6 and AR, this strongly supports the notion that these tumors have opposing gene expression patterns and should be considered a distinct subset of patients. APUC-6 exhibited distinct outcomes as compared to AR-high tumors. In prostate tumor biopsies (Caris cohort), patient

tumors with high APUC-6 had improved OS as compared to low APUC-6 (HR=0.515, 95% CI=0.442-0.599, p-value <0.0001). In the same subset of tumors, this effect was more robust compared to tumors with high expression of ESR1, ESR2, and PGR (HR=0.744, 95% CI=0.648-0.854, p-value <0.0001). Interestingly, AR-high tumors

exhibited the opposite outcomes (HR=1.96, 95% CI=1.704-2.253, p-value <0.0001) in these samples.

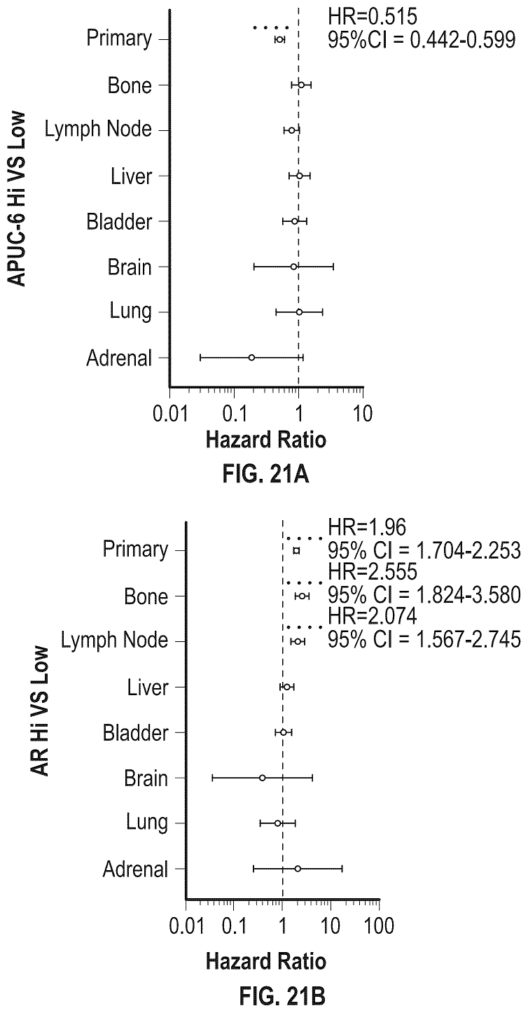

FIGS. 21A and 21B are graphs of example hazard ratio for different biopsy sites. When we examined the aggregate of all metastatic tumors or bone metastasis, APUC-6 high tumors no longer exhibited significant differences in outcomes (FIGS. 21A and 21B) As shown in FIGS. 21A and 21B, APUC-6 high and AR high tumors have distinct clinical outcomes. OS is shown for APUC-6 high or AR high PC tumors based on biopsy site. In this setting, patients with AR high tumors had worse outcomes when considering all prostate tumor biopsies (HR=2.0, 95% CI=1.7-2.3, p-value <0.0001) or bone (HR=2.6, 95% CI=1.8-3.6, p-value <0.0001) and lymph node metastasis (HR=2.1, 95% CI=1.6-2.7, p-value <0.0001).

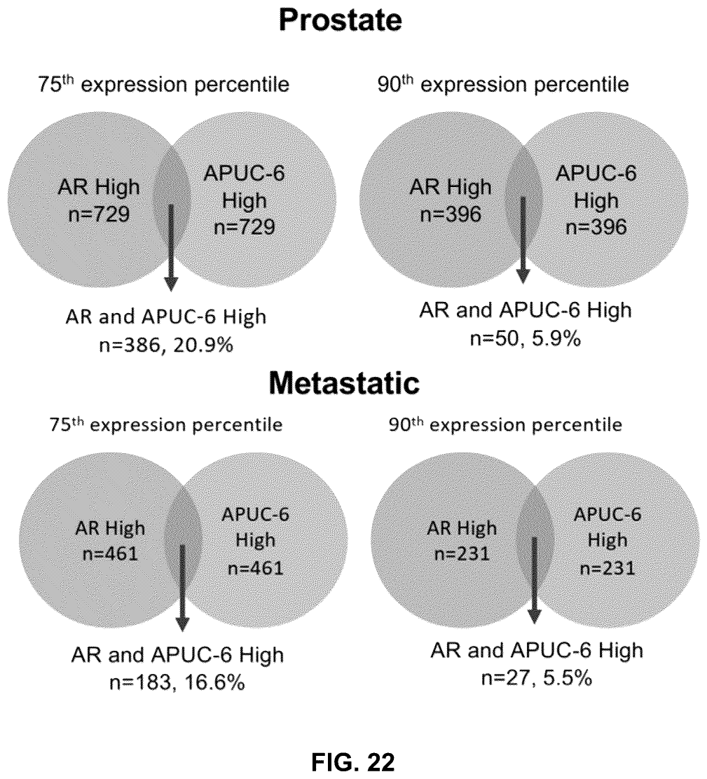

FIG. 22 includes Venn diagrams of example co-expressions for different tissues at different expression percentiles. Venn diagrams showing co-expression of AR

high and APUC-6 high prostate and metastatic (top and bottom, respectively) biopsies using two percentile thresholds-above 75th percentile and 90th percentile of target gene(s) expression. Similar to the SU2C/PCF pilot study, high expression of AR and APUC-6 genes was largely mutually exclusive in the Caris dataset, and only a minority of prostate or metastatic samples (5.9% and 5.5% respectively) shared elevated expression of AR and APUC-6 genes (FIG. 22).

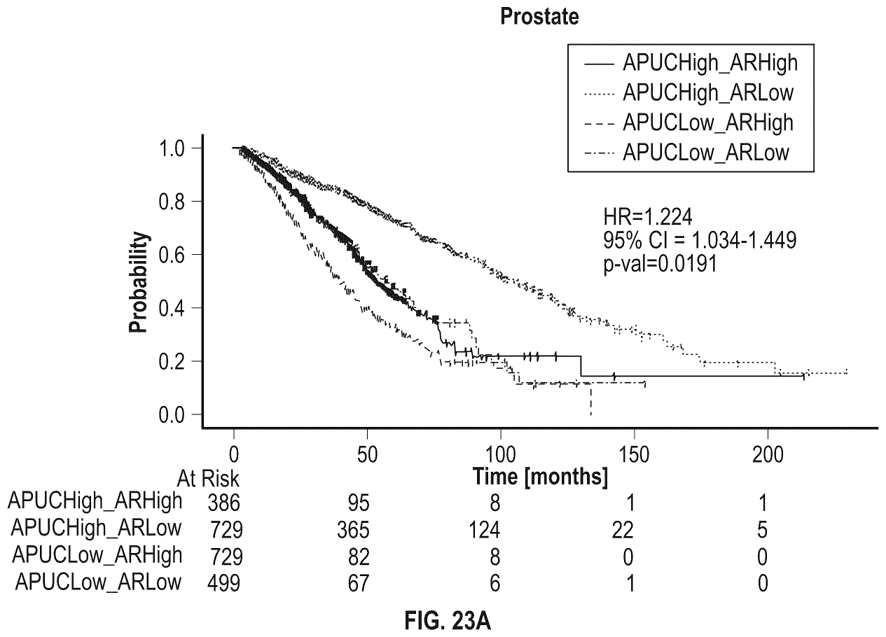

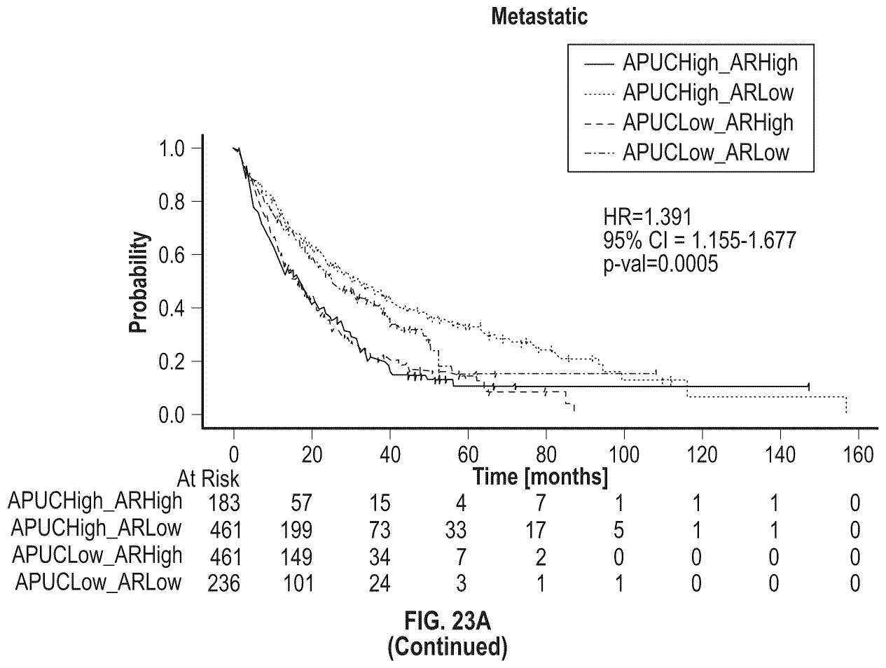

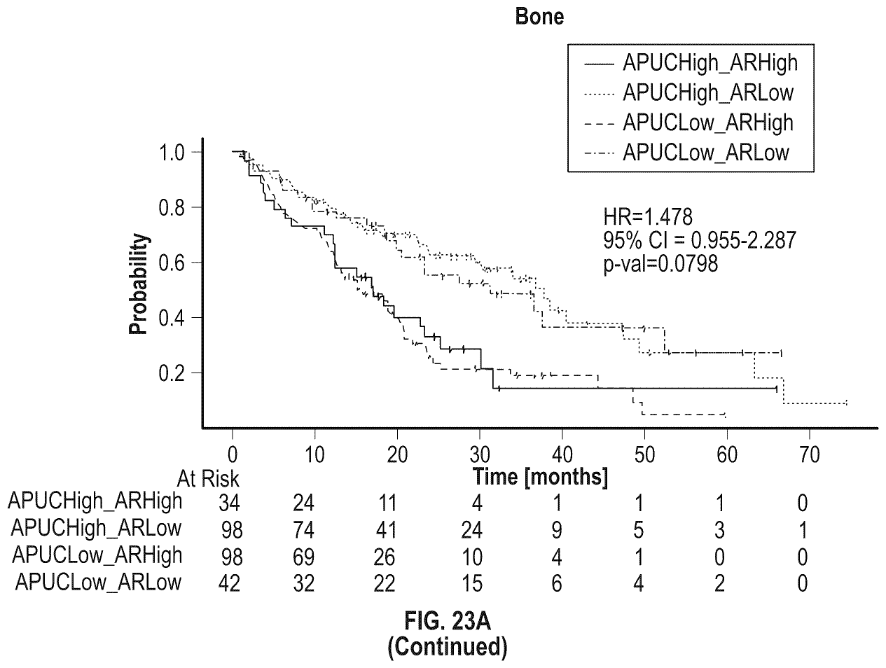

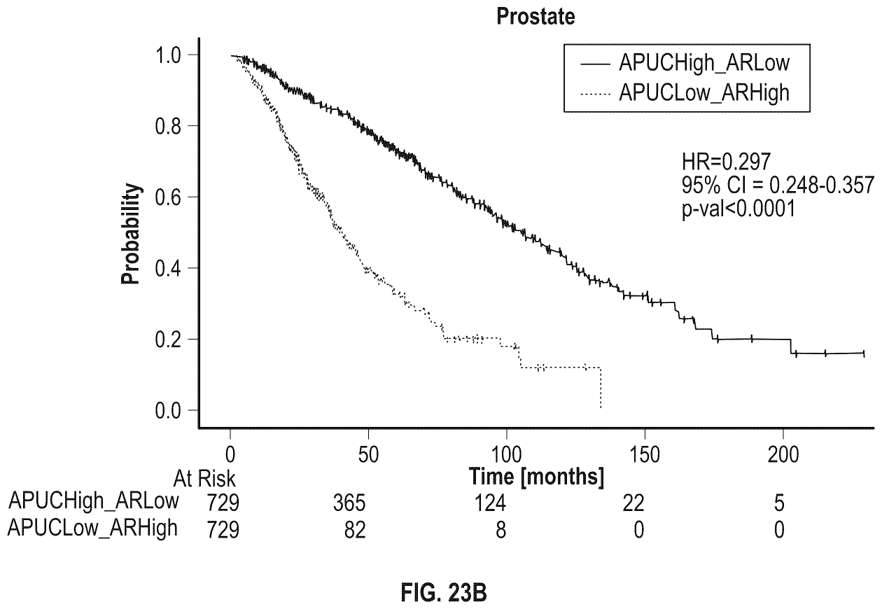

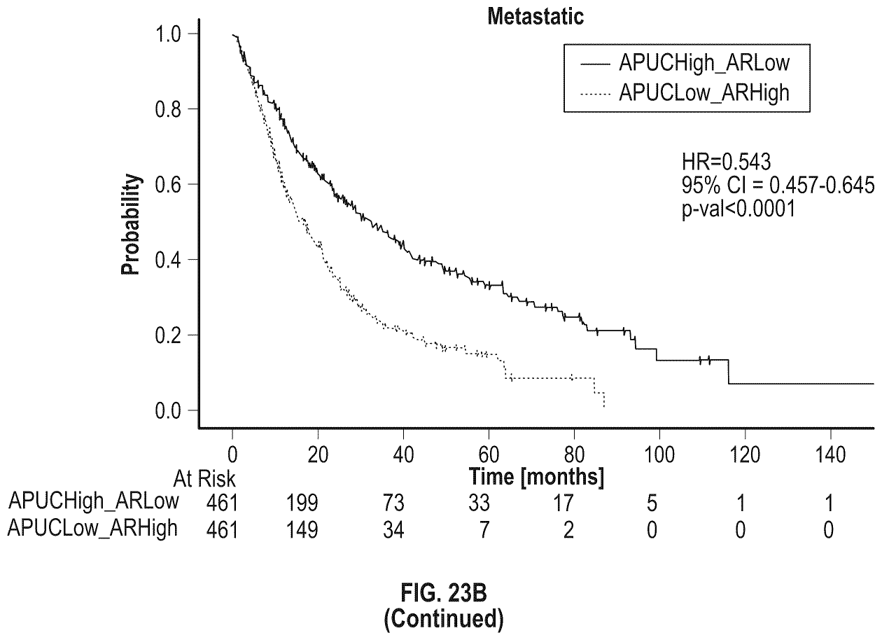

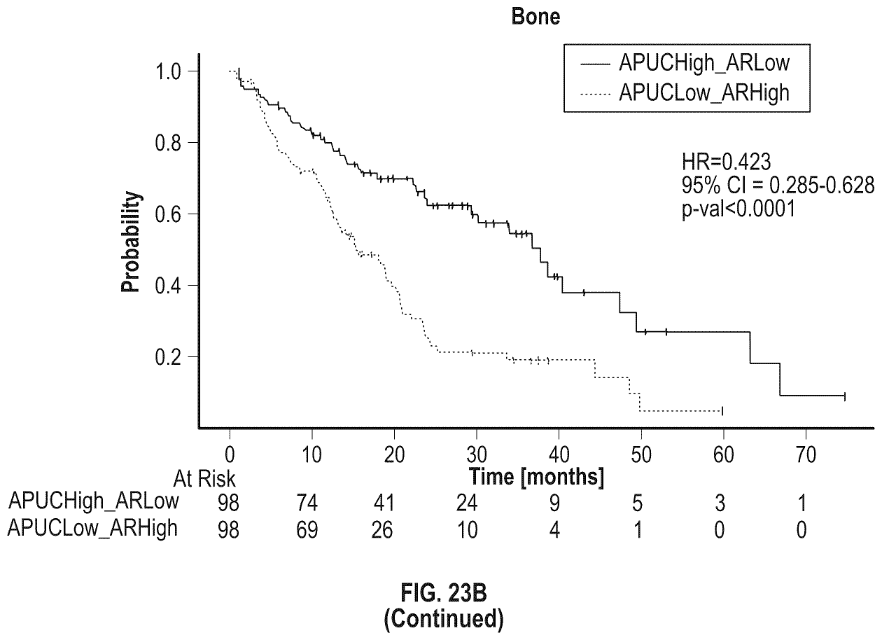

FIGS. 23A and 23B are graphs of overall survival probabilities for prostate cancer tumors based on biopsy site. As shown in FIGS. 23A and 23B, OS is shown for APUC-6 and AR expression status (4 combinations; APUC-6 high/AR high, APUC-6 high/AR low, APUC-6 low/AR high, APUC-6 low/AR low) across PC tumors based on biopsy site. D. OS is shown by APUC-6 and AR expression status (2 combinations; APUC-6 high/AR low versus APUC-6 low/AR high) across PC tumors based on biopsy site.

Using the knowledge from FIG. 22, 4 patient groups were established based on AR and APUC-6 expression (APUC-6 high/AR high, APUC-6 high/AR low, APUC-6 low/AR high, APUC-6 low/AR low) across prostate, metastatic, and bone biopsies (FIG. 23C). In all analyses, patients with the APUC-6 high/AR low tumors (orange line) had the best OS, whereas patients with APUC-6 low/AR high tumors (green) had the worst outcomes. When selected for patients with APUC-6 high/AR low and APUC-6 low/AR tumors and directly compared these groups, patients with APUC-6 high/AR low PC had significantly longer OS compared to patients with APUC-6 low/AR high PC for each tumor site (prostate biopsies, HR=0.297, 95% CI=0.248-0.357, p-value <0.0001; metastatic biopsies, HR=0.543, 95% CI=0.457-0.645, p-value <0.0001; Bone, HR=0.423, 95% CI=0.285-0.628, p-value <0.0001), as shown in FIG. 23D. Altogether, APUC-6 and AR overexpression status can be used to define two distinct subsets of PCs with different outcomes based on analysis using ALAN as described.

As described in the prostate cancer example, a subset of 6 APUC genes acted together to define a subset of PC that is uniquely driven by APUC variation and is phenotypically distinct from other aberrations, including those of the Androgen receptor and may implicate other steroid receptors beyond the AR. Deploying the novel ALAN computational approach captured 6 APUC genes (HSD3B1, HSD3B2, CYP11A1, CYP11B1, CYP3A43, CYP171A1) that, when analyzed collectively, exhibited consistent and robust associations with outcome in prostate or metastatic PC. Notable among these critical 6 genes is CYP171A1, the target of Abiraterone, CYP11A1, the target of opevesostat, and HSD3B1, which has already shown to have established impacts on patient outcomes. Altogether, these data suggest the existence of a new subset of prostate cancer that is uniquely dependent on androgen production, uptake, and conversion and is distinct from those with AR aberrations or AR high activity.

Several key observations merit further discussion and evaluation. When 7,083 tumor samples were examined from the prostate and metastatic PCs analyzed by clinical grade genomic tests by Caris, these genes consistently exhibited robust co-expression. The 6 APUC genes (APUC-6) were not associated with the expression of AR, AR-V7, or AR activity, were enriched with a basal transcriptional profile (as compared to luminal) but did not demonstrate NEPC signatures. Across all datasets, a consistent positive association of APUC-6 genes with each other as well as ESR1, ESR2, and PGR was detected.

Using both public and Caris datasets, the process could stratify two distinct subsets of PC patients based on AR or APUC-6 expression, in which APUC-6 high PCs had improved outcomes. AR overexpression was expectedly associated with worse

outcomes. This supports a deeper investigation into the effects of ER signaling in PC patients, which has been reported to regulate circulating testosterone levels or modulate key signaling pathways such as MYC in PC models.

Singular germline variants of APUC genes are known to regulate AR signaling. Most notably, 3βHSD1 function is enhanced by a missense-encoding variant (1245AàC), commonly detected in PC patients. PC patients with the HSD3B1 (1245C) allele experience unfavorable rates of both progression-free, metastasis-free, and overall survival from prostate cancer due to the enhancement of a missense-encoding variant that drives ADT resistance. The APUC-6 genes include HSD3B1 and appear to exhibit co-expression in essentially all major biopsy sites. Yet these APUC-6 high tumors instead exhibited reduced AR and AR-V7 expression as well as AR signatures and importantly reflected tumors with better OS. In explaining these observations, the convergent behavior of APUC-6 genes was specifically observed in metastatic PCs, or PC patients with a clinical condition that merited further genomic testing. This patient population is more likely to have high-volume disease, to have already progressed on ADT, or to present with intrinsic resistance. It is thus surprising and important to discover that APUC-6 represents a patient population with better OS with potentially unique vulnerabilities especially in the metastatic disease setting. Related to this, differences in the association of APUC-6 genes were detected when comparing the samples from TCGA to metastatic PCs and prostate biopsies from the Caris dataset. TCGA samples are exclusively primary prostate tumors, in which fewer patients in this cohort eventually develop metastasis. These findings indicate that APUC-6 genes, including HSD3B1, may have a distinct effect in patients with high-volume disease compared to localized or untreated disease. For example, that the adrenal-permissive HSD3B1 variant was not associated with clinical outcomes in the setting of high-volume disease 15. Altogether, it is important to recognize that APUC genes, particularly the 6 resulting from our analysis, may hold the greatest impact in treatment resistance as opposed to localized or untreated disease, a hypothesis that requires further study. This analysis may not be able to make definitive statements implicating this network of enzymes in tumorigenesis of localized disease.

While APUC-6 genes were associated with reduced AR activity in late-stage PC, we found exceptions to this observation. As tumor sites were examined within the 22 adrenal metastasis samples, it was clear that HSD3B1 and CYP3A43 exhibited robust association with AR expression (R=0.7 and 0.56), whereas the rest of the APUC-6 genes did not. This suggests that there are tissue-specific effects in the adrenal metastatic PCs that lead to distinct signaling mechanisms as compared to other metastatic sites. Outside of the APUC-6 genes, the analysis through ALAN indicated that in metastatic PC, HSD17B10, SRD5A3, and SULT2B1, unlike APUC-6 genes, exhibited positive associations with AR. This indicates that APUC genes other than the 6 we have examined may still regulate AR activity and thus could still be considered adrenal permissive. Relevant to our nomination of APUC-6, there were also other APUC genes that exhibited positive correlations with one or more of the 6 APUC genes. Particularly, LHCGR and HSD17B3 had positive associations with several APUC-6 genes as well as ESR1,

ESR2, and PGR. Altogether, future studies should continue to investigate other APUC genes and their regulation of hormone signaling.

Additional clinical implications of APUC-6 high tumors are considerable, including targeting of cell surface receptors. B7-H3 expression may be driven by AR signaling. PSMA, the target of 177Lu-PSMA-617 and other approaches under investigation, is AR-dependent. In APUC-6 tumors, we indeed found a decreased differential expression of B7-H3 and PSMA expression. In the same analysis, APUC-6 genes had significant differences in the expression of DLL336 and CEACAM537, both biomarkers and clinical targets implicated in advanced PCs with neuroendocrine-like

features. Notably, when examining the enrichment of the same genes with respect to AR-high tumors, the results were entirely opposite. Here APUC-6 tumors had increased basal-like signatures but not the neuroendocrine signature. Additionally, none of these APUC-6 tumors are associated with neuroendocrine histology. The mechanism and implications of these observations remain unclarified. Future studies may consider utilizing the APUC-6 genes to make decisions of therapeutic strategies, such as stratification in clinical studies and integration as a biomarker for selection into or away from novel therapies targeting cell surface molecules and enzymatic pathways. This provides a shorter path towards translation as compared to developing specific inhibitors against the enzymatic activity of APUC genes, which regulate ligand production for hormone receptors.

Finally, it is notable that one of the significant APUC genes was CYP11A1, the most proximal adrenal enzyme that converts cholesterol to pregnenolone, catalyzing the first and rate-limiting step of all steroid biosynthesis. This finding may be of clinical relevance due to the recent development of opevesostat (formerly MK-5684, or ODM-208), an oral selective inhibitor of CYP11A1, which has shown preliminary evidence of clinical activity in antiandrogen-refractory mCRPC patients. In a recent study, opevesostat produced PSA responses in a significant proportion of mCRPC patients but especially in those harboring activating AR ligand-binding domain mutations (PSA response rate, 38/64=59%). Interrogating the intratumoral expression of CYP11A1 (at the protein or transcript level) in these patients may also potentially refine our understanding of who may benefit most (or least) from this novel pan-steroid synthesis inhibitor.

The statistical analyses included are univariate analyses and causal relationships that may have not been established. For examination of the Caris POA dataset, the biopsy location does not always reflect disease stage or tumor grade as may be the case for the primary tumor biopsies. The data has been organized based on self-reported race, whereby certain patients were uncharacterized and excluded from analysis.

Of the many APUC genes that have been associated with the activation of AR, this example found that 6 (HSD3B1, HSD3B2, CYP11A1, CYP11B1, CYP17A1, CYP3A43) exhibit robust association in prostate and metastatic tumors and were mutually exclusive with heightened AR activity. Conversely, APUC-6 genes were instead associated with the expression of alternative hormone receptors ESR1, ESR2, and PGR. Prostate tumors with elevated expression of APUC-6 genes exhibited distinct outcomes as compared to those that were AR high and therefore represent a distinct subset of PC patients. Given that WTS is prevalently used for genomic testing, one should evaluate the expression of APUC-6 genes as well as ESR1, ESR2, and PGR in disease management of patients with prostate cancer.

Methods and materials. This study evaluating genes related to prostate cancer was conducted in accordance with the guidelines of the Declaration of Helsinki, Belmont Report, and U.S. Common Rule. In keeping with 45 CFR 46.101 (b), this study was carried out using retrospective deidentified clinical data. Therefore, patient consent was waived, and this study was considered exempt at each institutional review board.

Specimens. We queried the Caris Life Sciences database to assess molecular alterations and related survival outcomes of 7,083 prostate tumor biopsies.

Comprehensive molecular profiling was performed in a CLIA/CAP/ISO15189 certified clinical laboratory (Caris Life Sciences, Phoenix, AZ, USA). Samples annotated as prostate includes mixture of tumors from patients, in which some have metastatic disease but the biopsy was performed on the primary site. Metastatic samples were annotated based on biopsy location (bone, lymph node, liver, bladder, lung, and adrenal gland).

Sex as a biological variable. This study included the analysis of genomic data from human patients. Due to the nature of prostate cancer affecting males, only male patients were included in this study. However, we do anticipate that this study is relevant to more than one sex since, as discussed throughout, genes involving female steroid hormone receptors are associated with APUC-6. These SHR genes are known drivers of breast and ovarian cancer, however, the role of APUC-6 in those settings requires further investigation and was out of scope for this example.

Whole Transcriptome Sequencing (WTS) for Caris POA data. Qiagen RNA FFPE tissue extraction kit was used for extraction. RNA quality and quantity were determined using the Agilent TapeStation (RRID: SCR_019547). Biotinylated RNA baits were hybridized to the synthesized and purified cDNA targets, and the bait-target complexes were amplified in a post-capture PCR reaction. The Illumina NovaSeq 6500 was used to sequence the whole transcriptome from patients to an average of 60M reads. Raw data was demultiplexed by the Illumina Dragen BioIT accelerator, trimmed, counted,

removed of PCR-duplicates, and aligned to the human reference genome hg19 by the STAR aligner. For transcription counting, transcripts per million molecules were generated using the Salmon expression.

Public Dataset Preprocessing. Other than the Caris POA cohort data, all RNA-seq data were downloaded from public resources that can be found in the Data Availability section below. Processing was done to ensure each dataset was in TPM before analyses began and each dataset required distinct pre-processing depending on the starting format. TCGA Prostate. TCGA raw read counts and associated transcript length information were downloaded using the TCGAbiolinks (version 2.30.4, project=‘TCGA-PRAD’) R package. We then subset the raw reads to include only protein-coding genes and converted to TPM, which accounted for within-sample normalization based on read depth. SU2C/PCF 2019. SU2C 2019 FPKM data was downloaded from cBioPortal and then converted to TPM. The SU2C TPM data were further natural log-transformed for downstream analyses. The AR-V7 variant spliced reads per million expression data, AR score, and NEPC score were extracted directly from the clinical data file associated with the SU2C dataset downloaded from cBioPortal with no further modifications. GTEx Prostate. The GTEx dataset was downloaded directly from the web the portal and was already in TPM so no further conversion was necessary. Genes with zero expression across all samples were removed from the data and duplicate gene names were made unique.

ALAN analyses. This technique included performance of ALAN and depicted outputs as based on our prior study. As the units from all public resources were distinct, we converted all input RNA-seq data into TPM (See public dataset preprocessing), which accounted for within-sample normalization based on read depth. The 21 APUC genes were pulled from this output and visualized in 3 separate heatmaps, which employed unsupervised hierarchical clustering to group the genes in FIG. 9B. ALAN profiles for each of the APUC-6 genes were extracted and shown in the form of violin plots in FIG. 10A. Uniform Manifold Approximation and Projection (UMAP) was applied on the SU2C cohort ALAN outputs with the default parameters in FIG. 14.

Signature Scoring and Stratification. Gene sets were utilized from previously defined single cell RNA-seq signatures for Luminal and Basal subtypes. For the SU2C pilot study, the APUC-6, Luminal, and Basal scores were calculated by taking the sum of the log (TPM+1) expression of their respective gene sets in each sample in the cohort (n=208). These scores were then scaled from 0 to 100 across all samples. Samples with APUC-6 scores in the top 25th percentile and bottom 25th percentile were used to classify the APUC-high (n=52) and APUC-low (n=52) patients respectively. The AR and NEPC scores along with AR-V7 expression were previously generated in the original study and were downloaded from cBioPortal (clinical data file). For scoring tumors from the Caris database, Z-scores for genes were determined based on expression levels across patients. The average of Z-scores for either the 6 APUC genes or ESR1/2, and PGR were then utilized to group patient groups for subsequent analysis. This included stratification for APUC-6 high/AR high, APUC-6 high/AR low, APUC-6 low/AR high, and APUC-6 low/AR low patient groups, which were only generated in the Caris dataset to facilitate a more fine-grained OS analysis.

Genome-scale ORF screen analysis. This example analyzed a previously published genome-scale ORF screen that was performed in LNCaP cells. Specifically, we compared the experimental arms conducted in control media (FCS) with androgens and androgen stripped media (CSS) containing enzalutamide for each gene. The Z-scores of APUC-6 genes were averaged as compared to all other ORFs to reflect the relative effects of each group of genes on cell proliferation after 25 days in culture.

GSEA Analysis. Gene Set Enrichment Analysis (GSEA) 30 was conducted on all PC samples from the Caris POA cohort to analyze 50 Hallmark signatures based on APUC-6 high or AR high status. The enrichment plots were generated as output

along with net enrichment scores (NES) and false discovery rate (FDR) for each signature based on 1000 permutations. The process examined the relative rank of all genes based on the differential expression upon grouping samples based on APUC-6 or AR expression status.

Survival analysis. We queried the de-identified real-world evidence (RWE) outcomes dataset from the Caris Life Sciences Precision Oncology Alliance (POA) registry and insurance claims data. RWE overall survival (OS) was defined as date of

treatment initiation (day 0) to either the date of death or last contact in the insurance claims repository. As previously reported, patient death was assumed for any patient without a claim for more than 100 days, which holds true for more than 95% of patients with a recorded death in the National Death Index (NDI). Cox proportional hazard ratios were calculated for each comparison group and significance was determined as p values of <0.05 using the log-rank statistic.

Statistics. Statistical significance was determined using X2 and Mann-Whitney U tests with corrections for multiple comparisons where appropriate using the Benjamini-Hochberg method to control the false discovery rate (FDR) at a significance level of α=0.05. For determination of statistical significance in signature scoring, t-test was used when data was normally distributed where Wilcoxon's was used when data was not normally distributed as was the case for some comparisons in FIG. 12C. Differential gene expression was analyzed using the Limma R package (q<0.001, logFC >1.5, −log 10 FDR >20). Differential gene expression was analyzed by the Mann-Whitney U test for prostate tumors from Caris. APUC6 Q4 vs Q1, AR Q4 vs Q1 were tested.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media, which includes any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable storage medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

What is claimed is:

1. A system comprising:
a data repository configured to store molecular abundance values for a plurality of samples;
processing circuitry configured to:
receive, from the data repository, one or more matrices comprising at least one molecular abundance value for each sample of the plurality of samples;
generate a first correlation matrix by at least performing a first statistical correlation operation between every pair of columns within the one or more matrices;
generate a second correlation matrix by at least performing a second statistical correlation operation between every pair of columns within the first correlation matrix;
determine, based on the second correlation matrix, a metric indicative of one or more gene ecosystems for one or more tissue types of at least one sample of the plurality of samples; and
output, for display, the metric indicative of the one or more gene ecosystems.

2. The system of claim 1, wherein each row of each matrix of the one or more matrices corresponds to molecular abundance values corresponding to one sample, and wherein different columns of each matrix of the one or more matrices corresponds to different types of molecular abundance values.

3. The system of claim 1, wherein a first method of the first statistical correlation operation is different than a second method of the second statistical correlation operation.

4. The system of claim 1, wherein the first method comprises a rank-based Spearmans's correlation, and wherein the second method comprises a Pearson's correlation.

5. The system of claim 1, wherein the first correlation matrix identifies one or more gene profiles from the molecular abundance values of the one or more matrices.

6. The system of claim 5, wherein the second correlation matrix identifies a the one or more gene ecosystems from the one or more gene profiles of the first correlation matrix.

7. The system of claim 1, wherein the processing circuitry is further configured to determine a gene profile correlation between two gene profiles from individual cells of the second correlation matrix.

8. The system of claim 1, wherein the processing circuitry is further configured to determine a gene network correlation between two or more genes of the plurality of samples based on all rows of the second correlation matrix and two or more columns of the second correlation matrix corresponding to the two or more genes.

9. The system of claim 1, wherein the processing circuitry is further configured to determine gene network signatures for any genes having correlation scores above 0.7 from the second correlation matrix.

10. The system of claim 1, wherein the processing circuitry is further configured to determine a network score for the one or more gene ecosystems from any gene network signatures for any genes having correlation scores above 0.7.

11. The system of claim 1, wherein the processing circuitry is further configured to determine, based on the metric indicative of one or more gene ecosystems, a diagnosis of a condition associated with at least one sample of the plurality of samples.

12. The system of claim 1, wherein the processing circuitry is further configured to determine, based on the metric indicative of one or more gene ecosystems, a treatment plan for a condition associated with at least one sample of the plurality of samples.

13. The system of claim 1, wherein the at least one molecular abundance value comprises at least one of a gene expression value, a gene count, or a protein abundance value.

14. A method comprising:
receiving, by processing circuitry and from a data repository configured to store molecular abundance values for a plurality of samples, one or more matrices comprising at least one molecular abundance value for each sample of the plurality of samples;
generating, by the processing circuitry, a first correlation matrix by at least performing a first statistical correlation operation between every pair of columns within the one or more matrices;
generating, by the processing circuitry, a second correlation matrix by at least performing a second statistical correlation operation between every pair of columns within the first correlation matrix;
determining, by the processing circuitry and based on the second correlation matrix, a metric indicative of one or more gene ecosystems for one or more tissue types of at least one sample of the plurality of samples; and
outputting, by the processing circuitry and for display, the metric indicative of the one or more gene ecosystems.

15. The method of claim 14, wherein each row of each matrix of the one or more matrices corresponds to molecular abundance values corresponding to one sample, and wherein different columns of each matrix of the one or more matrices corresponds to different types of molecular abundance values.

16. The method of claim 14, wherein a first method of the first statistical correlation operation is different than a second method of the second statistical correlation operation.

17. The method of claim 14, wherein the first method comprises a rank-based Spearmans's correlation, and wherein the second method comprises a Pearson's correlation.

18. The method of claim 14, wherein the first correlation matrix identifies one or more gene profiles from the molecular abundance values of the one or more matrices.

19. The method of claim 14, further comprising determining a gene network correlation between two or more genes of the plurality of samples based on all rows of the second correlation matrix and two or more columns of the second correlation matrix corresponding to the two or more genes.

20. A non-transitory computer-readable medium comprising instructions configured to, when executed, causes processing circuitry to:
receive, from a data repository configured to store molecular abundance values for a plurality of samples, one or more matrices comprising at least one molecular abundance value for each sample of the plurality of samples;
generate a first correlation matrix by at least performing a first statistical correlation operation between every pair of columns within the one or more matrices;
generate a second correlation matrix by at least performing a second statistical correlation operation between every pair of columns within the first correlation matrix;
determine, based on the second correlation matrix, a metric indicative of one or more gene ecosystems for one or more tissue types of at least one sample of the plurality of samples; and
output, for display, the metric indicative of the one or more gene ecosystems.
